(19) United States
(12) Patent Application Publication
(10) Pub. No.: US 2023/0368866 A1
(43) Pub. Date: Nov. 16, 2023

(54) ADAPTIVE NEURAL NETWORK FOR NUCELOTIDE SEQUENCING

(71) Applicant: Illumina Software, Inc., San Diego, CA (US)

(72) Inventor: Gavin Derek Parnaby, Laguna Niguel, CA (US)

(21) Appl. No.: 18/315,142

(22) Filed: May 10, 2023

Related U.S. Application Data
(60) Provisional application No. 63/364,486, filed on May 10, 2022.

(51) Int. Cl. G16B 30/00; G06N 3/04; G06N 3/08; G16B 40/20

(52) U.S. Cl. CPC G16B 30/00; G06N 3/04; G06N 3/08; G16B 40/20

(57) ABSTRACT

This disclosure describes methods, non-transitory computer readable media, and systems that can configure a field programmable gate array (FPGA) or other configurable processor to implement a neural network and train the neural network using the configurable processor by modifying certain network parameters of a subset of the neural network’s layers. For instance, the disclosed systems can configure a configurable processor on a computing device to implement a base-calling-neural network (or other neural network) that includes different sets of layers. Based on a set of images of oligonucleotide clusters or other datasets, the neural network generates predicted classes, such as by generating nucleobase calls for oligonucleotide clusters. Based on the predicted classes, the disclosed systems subsequently modify certain network parameters for a subset of the neural network’s layers, such by modifying parameters for a set of top layers.

### CROSS-REFERENCE TO RELATED APPLICATIONS

The present application claims the benefit of, and priority to, U.S. Provisional Application No. 63/364,486, entitled “ADAPTIVE NEURAL NETWORK FOR NUCELOTIDE SEQUENCING,” filed on May 10, 2022. The aforementioned application is hereby incorporated by reference in its entirety.

### BACKGROUND

In recent years, biotechnology firms and research institutions have improved hardware and software, such as neural networks, for sequencing nucleotides and determining nucleobase calls for genomic samples. For instance, some existing sequencing machines, on-device software, and sequencing-data-analysis software (together “existing sequencing systems”) determine individual nucleobases within sequences by using conventional Sanger sequencing or by using sequencing-by-synthesis (SBS) methods. When using SBS, existing sequencing systems can monitor many thousands of oligonucleotides being synthesized in parallel from templates to predict nucleobase calls for growing nucleotide reads. For instance, a camera in many existing sequencing systems captures images of irradiated fluorescent tags incorporated into oligonucleotides. After capturing such images, some existing sequencing systems use a neural network to process the image data from the camera and determine nucleobase calls for nucleotide reads corresponding to the oligonucleotides. Based on the nucleobase calls for such reads, existing systems further utilize a variant caller to identify variants, such as single nucleotide polymorphisms (SNPs), insertions or deletions (indels), or other variants within a genomic sample. Despite recent technologies that have developed neural networks to generate nucleobase calls from image or other data, existing sequencing systems often use neural networks that are computationally costly to both train and execute as well as rigidly fixed during execution on local sequencing machines without customization.

As just suggested, some existing sequencing systems both train and execute neural networks that consume inordinate computer processing and memory. To train a deep neural network as a base caller, for instance, existing sequencing systems often process vast amounts of genomic data from reference genomes or other genomic databases. Such neural networks can take days or weeks to train on high-powered servers, where processing a single 100×100 pixel image consumes 500 or more million multiplication operations. Because such deep neural networks often include many layers (such as ten or more convolutional layers) and must tune parameters for such layers, existing systems that tune a full set of initialized parameters (without transfer learning) can drag training on for weeks or months. To ensure sufficient computer processing and memory bandwidth, existing systems often run training iterations with one or more graphics processing units (GPUs) or central processing unit (CPUs) to train weights or other parameters of a neural network for base calling, where GPU cores execute on multiple threads while waiting for sufficient memory to proceed. In some cases, existing sequencing systems also train different versions of deep neural networks (sometimes with different genomic datasets) and select a highest performing network to deploy on sequence platforms in laboratories or other locations.

After training such a neural network, existing sequencing systems often deploy a fixed-parameter version of the neural network on sequencing machines to predict nucleobase calls for genomic samples-without further training. When deployed on such sequencing machines, however, fixed-parameter neural networks do not adapt to mechanical and biochemical environments that differ from the initial sequencing machines (e.g., in a data center) used to train the neural network. During a sequencing run, for instance, a sequencing machine may use a camera with different calibrations, a nucleotide-sample slide with different biochemistry or reagents, or different internal device temperatures than those used for the initial sequencing machines used for training.

Further complicating differences, the on-site sequencing machine may determine nucleobase calls for genomic samples with a distribution of nucleobase types that varies from genomic samples or reference genomes used to train the initial sequencing machines. Overtime, as the environment of an on-site sequencing machine and nucleotide-sample slide changes-without changes to fixed parameters of a neural network-the neural network fails to account for changes to a camera’s focus, responsiveness to a camera’s field of view, lighting, phasing or pre-phasing rates, batch effects on reagents or other chemicals, or other biochemical or mechanical factors. Because a neural network with fixed parameters cannot adapt to such biochemical or mechanical changes, the network’s performance and sequencing machine’s performance degrades overtime in analyzing images of oligonucleotide clusters.

Because of the different biochemistry and machinery between sequencing machines-and the changing environment of onsite sequencing machines-existing sequencing systems can inaccurately analyze or detect signals from oligonucleotide cluster images for less accurate nucleobase calls using such fixed-parameter neural networks. Indeed, existing sequencing systems using the same fixed-parameter neural network to determine nucleobase calls can exhibit varying accuracies of image analysis or degrees of base-call accuracy-depending on the type and environment of the sequencing machine executing the neural network relative to the sequencing machine used for training the neural network. As such image-analysis accuracy and base-call accuracy can vary, some sequencing machines exhibit image-analysis accuracy or base-call accuracy below probabilistic base-call models that some neural networks are designed to outperform-thereby eliminating the accuracy gains of using the neural network.

These, along with additional problems and issues exist in existing sequencing systems.

### SUMMARY

This disclosure describes one or more embodiments of systems, methods, and non-transitory computer readable storage media that solve one or more of the problems described above or provide other advantages over the art. In particular, the disclosed system can configure a field programmable gate array (FPGA) or other configurable processor to implement a neural network and train the neural network using the configurable processor by modifying certain network parameters of a subset of the neural network’s layers. For instance, the disclosed systems can configure a configurable processor on a computing device to implement a base-calling-neural network (or other neural network) that includes different sets of layers. Based on a set of images of oligonucleotide clusters or other datasets, the neural network generates predicted classes, such as by generating nucleobase calls for oligonucleotide clusters. Based on the predicted classes, the disclosed systems subsequently modify certain network parameters for a subset of the neural network’s layers, such by modifying parameters for a set of top layers. By selectively modifying certain network parameters for a subset of neural network layers during (or as a consequence of) a sequencing run, in some embodiments, the disclosed systems train a base-calling-neural network to analyze oligonucleotide-cluster images and determine nucleobase calls with more computational efficiency and better accuracy than existing sequencing systems.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which an adaptive sequencing system can operate in accordance with one or more embodiments of the present disclosure.

FIG. 2A illustrates a schematic diagram of the adaptive sequencing system configuring a configurable processor to implement a neural network and train the neural network using the configurable processor by adjusting one or more network parameters of a subset of the neural network’s layers in accordance with one or more embodiments of the present disclosure.

FIG. 2B illustrates a schematic diagram of the adaptive sequencing system configuring a field programmable gate array (FPGA) to implement a base-calling-neural network and train the base-calling-neural network using the FPGA by adjusting one or more network parameters of a subset of the base-calling-neural network’s layers in accordance with one or more embodiments of the present disclosure.

FIG. 3 illustrates a schematic diagram of the adaptive sequencing system determines a gradient for adjusting one or more network parameters and modifies one or more network parameters corresponding to images or image regions based on the gradient in accordance with one or more embodiments of the present disclosure.

FIG. 4 illustrates the adaptive sequencing system training multiple neural networks using configurable processors based on a gradient or loss determined from predicted classes of a single neural network in accordance with one or more embodiments of the present disclosure.

FIGS. 5A-5B illustrate an example architecture of a base-calling-neural network and modifying certain network parameters for a subset of layers within the base-calling-neural network in accordance with one or more embodiments of the present disclosure.

FIG. 5C illustrates the adaptive sequencing system using a neural network to determine predicted classes for a target time period based on intermediate values corresponding to a different time period and generating additional predicted classes for the different time period based on the intermediate values in accordance with one or more embodiments of the present disclosure.

FIG. 6 illustrates a graph depicting multiplication operations (or “mults”) per image-patch operation for convolutional operations corresponding to different layers of a base-calling-neural network in accordance with one or more embodiments of the present disclosure.

FIG. 7 illustrates a graph depicting base-call error rates for a sequencing device running a first version of a base-calling-neural network with fixed network parameters and a sequencing device running a second version of a base-calling-neural network with network parameters adjusted by the adaptive sequencing system during a sequencing run in accordance with one or more embodiments of the present disclosure.

FIG. 8 illustrate series of acts for configuring a configurable processor to implement a neural network and train the neural network using the configurable processor by adjusting one or more network parameters of a subset of the neural network’s layers in accordance with one or more embodiments of the present disclosure.

FIG. 9 illustrate series of acts for configuring a configurable processor to implement a base-calling-neural network and train the base-calling-neural network using the configurable processor by adjusting one or more network parameters of a subset of the base-calling-neural network’s layers in accordance with one or more embodiments of the present disclosure.

FIG. 10 illustrates a block diagram of an example computing device in accordance with one or more embodiments of the present disclosure.

### DETAILED DESCRIPTION

This disclosure describes one or more embodiments of an adaptive sequencing system that can configure a field programmable gate array (FPGA) or other configurable processor to implement a base-calling-neural network and train the base-calling-neural network using the configurable processor by modifying certain network parameters of a subset of the neural network’s layers-while freezing other parameters of a different subset of the neural network’s layers. For instance, the adaptive sequencing system can configure a configurable processor on a sequencing device to implement a base-calling-neural network that includes a first set of layers and a second set of layers. For a target sequencing cycle of a sequencing run, the adaptive sequencing system can provide a set of images of oligonucleotide clusters to the base-calling-neural network and generate nucleobase calls for oligonucleotide clusters based on the set of images for the target sequencing cycle. Based on the nucleobase calls, the adaptive sequencing system subsequently uses the configurable processor to modify certain network parameters for the second set of layers while maintaining certain network parameters for the first set of layers.

As suggested above, the adaptive sequencing system can tune or customize certain network parameters of a base-calling-neural network that includes layers with network parameters initially trained by a different computing device. For instance, in some cases, a remote sequencing device (or a collection of remote sequencing devices) run training iterations with graphics processing units (GPUs) or central processing unit (CPUs) to train weights or other parameters of a base-calling-neural network based on training oligonucleotide-cluster images. After training an initial version of the base-calling-neural network, the adaptive sequencing system downloads or otherwise receives data representing the initial version of the base-calling-neural network and configures a FPGA of a sequencing device to implement the initial version of the base-calling-neural network. Alternatively, the adaptive sequencing system configures one or more application-specific integrated circuits (ASICs), application-specific standard products (ASSPs), or another configurable processor. Regardless of the configurable processor configured to implement the base-calling-neural network, the adaptive sequencing system can adjust network parameters for a subset of layers of the base-calling-neural network during sequencing cycles within a unique mechanical and biochemical environment of the sequencing device.

When running a version of the base-calling-neural network onsite on a sequencing device, for instance, the adaptive sequencing system can adjust certain network parameters for a second set of layers while maintaining certain network parameters for a first set of layers. In some cases, for instance, the adaptive sequencing system generates nucleobase calls for a target sequencing cycle and, based on a loss gradient or other cost and the nucleobase calls, modifies network parameters for a set of top layers and certain network parameters for a subset of bottom layers. In certain implementations, however, the adaptive sequencing system does not adjust certain network parameters for a different subset of bottom layers. In some embodiments of the base-calling-neural network, as part of or after a target sequencing cycle, the adaptive sequencing system modifies network parameters for both a set of temporal layers and a subset of spatial layers-but not for a different subset of spatial layers. By tuning network parameters for such a subset of layers, the adaptive sequencing system can efficiently customize a base-calling-neural network on a sequencing device’s FPGA or on another configurable processor.

In addition to selectively tuning layers of a base-calling-neural network during onsite training, in some embodiments, the adaptive sequencing system limits adjustments for other parameters or value calculations to efficiently train the base-calling-neural network onsite. For instance, in some embodiments, the adaptive sequencing system determines a gradient with a fixed-point range (rather than a floating-point range) based on nucleobase calls for a given sequencing cycle and modifies network parameters for a subset of layers according to the fixed-point-range gradient. In addition to an efficient gradient, in certain implementations, the adaptive sequencing system (i) determines nucleobase calls for oligonucleotide-cluster images of a target sequencing cycle based on intermediate values for additional oligonucleotide-cluster images corresponding to an additional sequencing cycle and (ii) generates additional nucleobase calls for the additional oligonucleotide-cluster images and the additional sequencing cycle based on the intermediate values and compressed channels-rather than redetermining the intermediate values during multiple sequencing cycles.

As indicated above, the adaptive sequencing system provides several technical advantages relative to existing sequencing systems by, for example, improving the computational efficiency and customization of training of a neural network as well as improving the analysis of oligonucleotide-cluster images (or other data) relative to existing sequencing systems. In some embodiments, the adaptive sequencing system improves the computational efficiency of training a neural network in the field or onsite. As noted above, some existing sequencing systems train complex neural networks with deep layer sets for base calling by adjusting a full set of initialized parameters each training iteration. Such conventional training with both forward and backward paths can consume weeks or months of computer processing for a neural network. By contrast, the adaptive sequencing system trains a neural network with a more computational efficient approach that adjusts a subset of network parameters using an FPGA or other configurable processor on a sequencing device or other onsite computing device. By configuring a configurable processor to implement a neural network and adjusting a subset of network parameters for a subset of the neural network’s layers, the adaptive sequencing system avoids the computational costs of adjusting a full set of parameters in each training iteration. Rather than adding neural network layers or otherwise adding complexity to a neural network to account for different sequencing devices, the adaptive sequencing system can tune parameters for a subset of layers during sequencing cycles-after the neural network’s layers have been initially trained on a different computing device-thereby avoiding the additional computational processing that taxes neural networks of existing sequencing systems. Similarly, in some embodiments, the adaptive sequencing system further simplifies computational processing by adjusting parameters based on a gradient with a fixed-point range rather than a floating-point range that can add time and additional computation in each training iteration.

In addition to improved computational efficiency, in certain implementations, the adaptive sequencing system customizes and flexibly modifies parameters of a neural network for a specific sequencing device or other computing device. As noted above, some existing sequencing systems train a fixed-parameter version of a neural network on sequencing machines and then deploy the same fixed-parameter version on a different, onsite sequencing machine. As the machinery or biochemistry of the onsite sequencing machine differs (or changes over time) from those devices used for training, the fixed parameters no longer reflect the sensors, reagents, temperatures, or other environmental aspects contributing to a sequencing device’s analysis of oligonucleotide-cluster images. By contrast, in some embodiments, the adaptive sequencing system adjusts and customizes parameters for a subset of network layers of a neural network as part of (or after) a target sequencing cycle of a sequencing device. The network parameters of a neural network can accordingly be modified by the adaptive sequencing system to be specific to (and a real-time reflection of) the sensors, mechanics, biochemistry, or other environmental factors of a sequencing device. Indeed, in some embodiments, the adaptive sequencing system is sequencing-device agnostic by customizing parameters of a base-calling-neural network to the unique machinery, biochemistry, sequencing runs, or user behavior for a sequencing device. As a base-calling-neural network analyzes oligonucleotide-cluster images and generates nucleobase calls for a given sequencing cycle, for instance, the adaptive sequencing system tunes parameters for the base-calling-neural network to improve image analysis and corresponding base calling.

Beyond improved computational efficiency or flexible customization, in some embodiments, the adaptive sequencing system trains a neural network to analyze images and determine predicted classes with more accuracy than existing sequencing systems or training systems. As noted above, some existing sequencing systems that run fixed-parameter neural networks on sequencing devices exhibit image-analysis accuracy or base-call accuracy below probabilistic base-call models that some neural networks are designed to outperform. In other words, the image-analysis accuracy and base-call accuracy of neural networks on sequencing devices can vary significantly depending on differences in machinery and biochemistry underlying images and can degrade over time. By contrast, the adaptive sequencing system can train a base-calling-neural network to better analyze oligonucleotide-cluster images and determine more accurate nucleobase calls. Indeed, the adaptive sequencing system can modify parameters of a base-calling-neural network to reflect the different machinery context or biochemistry context of an onsite sequencing machine, including, but not limited to, different types of sensors, reagents, temperatures, sequencing runs, or other environmental aspects contributing to a sequencing device’s analysis of oligonucleotide-cluster images-while improving the accuracy of analyzing oligonucleotide-cluster images and determining nucleobase calls. As shown and described below, in some embodiments, the adaptive sequencing system trains a base-calling-neural network to determine nucleobase calls with a lower error rate than a fixed-parameter base-calling-neural network. For a given sequencing cycle and/or over time, the adaptive sequencing system performs a unique and customized modification of network parameters to improve an accuracy of image analysis and base calling.

As illustrated by the foregoing discussion, the present disclosure utilizes a variety of terms to describe features and advantages of the adaptive sequencing system. As used herein, for example, the term “configurable processor” refers to a circuit or chip that can be configured or customized to perform a specific application. For instance, a configurable processor includes an integrated circuit chip that is designed to be configured or customized on site by an end user’s computing device to perform a specific application. Configurable processors include, but are not limited to, an ASIC, ASSP, a coarse-grained reconfigurable array (CGRA), or FPGA. By contrast, configurable processors do not include a CPU or GPU.

As mentioned, in some embodiments, the adaptive sequencing system uses a neural network to generate nucleobase calls or other predicted classes. The term “neural network” refers to a machine learning model that can be trained and/or tuned based on inputs to determine classifications or approximate unknown functions. For example, a neural network includes a model of interconnected artificial neurons (e.g., organized in layers) that communicate and learn to approximate complex functions and generate outputs (e.g., generated digital images) based on a plurality of inputs provided to the neural network. In some cases, a neural network refers to an algorithm (or set of algorithms) that implements deep learning techniques to model high-level abstractions in data. For example, a neural network can include a convolutional neural network (CNN), a recurrent neural network (e.g., an LSTM), a graph neural network, a self-attention transformer neural network, or a generative adversarial neural network.

In some cases, the adaptive sequencing system utilizes a base-calling-neural network to generate nucleobase calls based on images of oligonucleotide clusters on a nucleotide-sample slide. As used herein, the term “base-calling-neural network” refers to a neural network that generates one or more nucleobase calls. For example, in some cases, the base-calling-neural network is trained to generate one or more nucleobase calls indicating various probabilities or predictions of nucleobase calls for individual oligonucleotide clusters depicted in an image. As described below, in certain implementations, a base-calling-neural network processes a set of oligonucleotide-cluster images corresponding to different sequencing cycles to determine nucleobase calls for a target sequencing cycle.

Relatedly, the term “layer” or “neural network layer” refers to a collection of units or a unit of a neural network, such as a collection of one or more nodes or artificial neurons. For instance, a layer may include an input layer, a hidden layer, or an output layer of a neural network. In some cases, layers can be grouped together in different sets, such as a first set of layers and a second set of layers. For instance, “bottom layers” refer to layers relatively closer to an input layer, whereas “top layers” refer to layers relatively closer to an output layer. For instance, in some embodiments, a set of bottom layers includes neural network layers closer to an input layer than to an output layer, such as the initial four or five layers following an input layer from a neural network that comprises nine layers in between an input layer and an output layer. Conversely, a set of top layers includes neural network layers closer to an output layer than to an input layer, such as the three or four layers immediately before an output layer from a neural network that comprises nine layers in between an input layer and an output layer. As a further example, in some circumstances, layers can be grouped according to a type of analysis or function of the layers. A set of spatial layers, for instance, processes spatial or location-based information from data, whereas a set of temporal layers processes temporal or time-based information from data.

As further used herein, the term “predicted class” refers to a class or type determined by a neural network. For instance, a predicted class includes a probabilistic or binary classification output by a neural network, including probabilities or binary outputs corresponding to multiple classifications. A predicted class includes, but is not limited to, a nucleobase call, a font type, a facial classification, or an object type.

As further used herein, the term “nucleobase call” (or simply “base call”) refers to a determination or prediction of a particular nucleobase (or nucleobase pair) for an oligonucleotide (e.g., read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide-fragment read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or other base-call-output file-based on nucleotide-fragment reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, or a thymine (T) call.

As further used herein, the term “nucleotide-sample slide” refers to a plate or slide comprising oligonucleotides for sequencing nucleotide sequences from genomic samples or other sample nucleic-acid polymers. In particular, a nucleotide-sample slide can refer to a slide containing fluidic channels through which reagents and buffers can travel as part of sequencing. For example, in one or more embodiments, a nucleotide-sample slide includes a flow cell (e.g., a patterned flow cell or non-patterned flow cell) comprising small fluidic channels and short oligonucleotides complementary to binding adapter sequences. As indicated above, a nucleotide-sample slide can include wells (e.g., nanowells) comprising clusters of oligonucleotides.

As suggested above, a flow cell or other nucleotide-sample slide can (i) include a device having a lid extending over a reaction structure to form a flow channel therebetween that is in communication with a plurality of reaction sites of the reaction structure and (ii) include a detection device that is configured to detect designated reactions that occur at or proximate to the reaction sites. A flow cell or other nucleotide-sample slide may include a solid-state light detection or imaging device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDS)). The excitation light may provide emission signals (e.g., light of a wavelength or wavelengths that differ from the excitation light and, potentially, each other) that may be detected by the light sensors of the flow cell.

As further used herein, the term “sequencing cycle” refers to an iteration of adding or incorporating a nucleobase to an oligonucleotide representing or corresponding to a nucleotide sequence or an iteration of adding or incorporating nucleobases to oligonucleotides representing or corresponding to nucleotide sequences in parallel. In particular, a sequencing cycle can include an iteration of capturing and analyzing one or more images with data indicating individual nucleobases added or incorporated into an oligonucleotide or to oligonucleotides (in parallel) representing or corresponding to one or more nucleotide sequences. For example, in one or more embodiments, each sequencing cycle involves capturing and analyzing images to determine either single reads of DNA (or RNA) strands representing part of a genomic sample (or transcribed sequence from a genomic sample). In some cases the sequencing cycle includes incorporating a nucleobase corresponding to an indexing sequence, a sample genomic sequence, or another nucleotide sequence within a sample fragment library.

In some cases, each sequencing cycle involves a camera capturing an image of a nucleotide-sample slide or images of multiple sections (e.g., tiles) of the nucleotide-sample slide to generate image data of particular nucleobases added or incorporated into particular oligonucleotides, which are often grouped in clusters. Following the image capture stage, the adaptive sequencing system can remove certain fluorescent labels from incorporated nucleobases and perform another cycle until a nucleotide sequence has been completely sequenced.

As further used herein, the term “sequencing run” refers to an iterative process on a sequencing device to determine a primary structure of nucleotide sequences from a sample (e.g., genomic sample). In particular, a sequencing run includes cycles of sequencing chemistry and imaging performed by a sequencing device that incorporate nucleobases into growing oligonucleotides to determine nucleotide-fragment reads from nucleotide sequences extracted from a sample (or other sequences within a library fragment) and seeded throughout a nucleotide-sample slide. In some cases, a sequencing run includes replicating nucleotide sequences from one or more genome samples seeded in clusters throughout a nucleotide-sample slide (e.g., a flow cell). Upon completing a sequencing run, a sequencing device can generate base-call data in a file.

As just suggested, the term “base-call data” refers to data representing nucleobase calls for nucleotide-fragment reads and/or corresponding sequencing metrics. For instance, base-call data includes textual data representing nucleobase calls for nucleotide-fragment reads as text (e.g., A, C, G, T) along with corresponding base-call-quality metrics, depth metrics, and/or other sequencing metrics. In some cases, base-call data is formatted in a text file, such as a binary base call (BCL) sequence file or as a fast-all quality (FASTQ) file.

By contrast, the term “oligonucleotide-cluster image” or “image of oligonucleotide clusters” refers to a digital image of one or more oligonucleotide clusters. In particular, in some cases, an oligonucleotide-cluster image refers to a digital image of one or more light signals (e.g., captured as different signal intensities) from one or more oligonucleotide clusters on a nucleotide-sample slide. For instance, in one or more embodiments, an oligonucleotide-cluster image includes a digital image captured by a camera during a sequencing cycle of light emitted by irradiated fluorescent tags incorporated into oligonucleotides from one or more clusters on a nucleotide-sample slide.

As further used herein, the term “nucleotide-fragment read” (or simply “read”) refers to an inferred sequence of one or more nucleobases (or nucleobase pairs) from all or part of a sample nucleotide sequence (e.g., a sample genomic sequence, cDNA). In particular, a nucleotide-fragment read includes a determined or predicted sequence of nucleobase calls for a nucleotide sequence (or group of monoclonal nucleotide sequences) from a sample library fragment corresponding to a genome sample. For example, in some cases, a sequencing device determines a nucleotide-fragment read by generating nucleobase calls for nucleobases passed through a nanopore of a nucleotide-sample slide, determined via fluorescent tagging, or determined from a cluster in a flow cell.

As used herein, the term “phasing” refers to an instance of (or rate at which) labeled nucleotide bases are incorporated behind a particular sequencing cycle. Phasing includes an instance of (or rate at which) labeled nucleotide bases within a cluster are asynchronously incorporated behind other labeled nucleotide bases within a cluster for a particular sequencing cycle. In particular, during SBS, each DNA strand in a cluster extends incorporation by one nucleotide base per cycle. One or more oligonucleotide strands within the cluster may become out of phase with the current cycle. Phasing occurs when nucleotide bases for one or more oligonucleotides within a cluster fall behind one or more cycles of incorporation. For example, a nucleotide sequence from a first location to a third location may be CTA. In this example, the C nucleotide should be incorporated in a first cycle, T in the second cycle, and A in the third cycle. When phasing occurs during the second sequencing cycle, one or more labeled C nucleotides are incorporated instead of a T nucleotide. Relatedly, as used herein, the term “pre-phasing” refers to an instance of (or rate at which) one or more nucleotide bases are incorporated ahead of a particular cycle. Pre-phasing includes an instance of (or rate at which) labeled nucleotide bases within a cluster are asynchronously incorporated ahead other labeled nucleotide bases within a cluster for a particular sequencing cycle. To illustrate, when pre-phasing occurs during the second sequencing cycle in the example above, one or more labeled A nucleotides are incorporated instead of a T nucleotide.

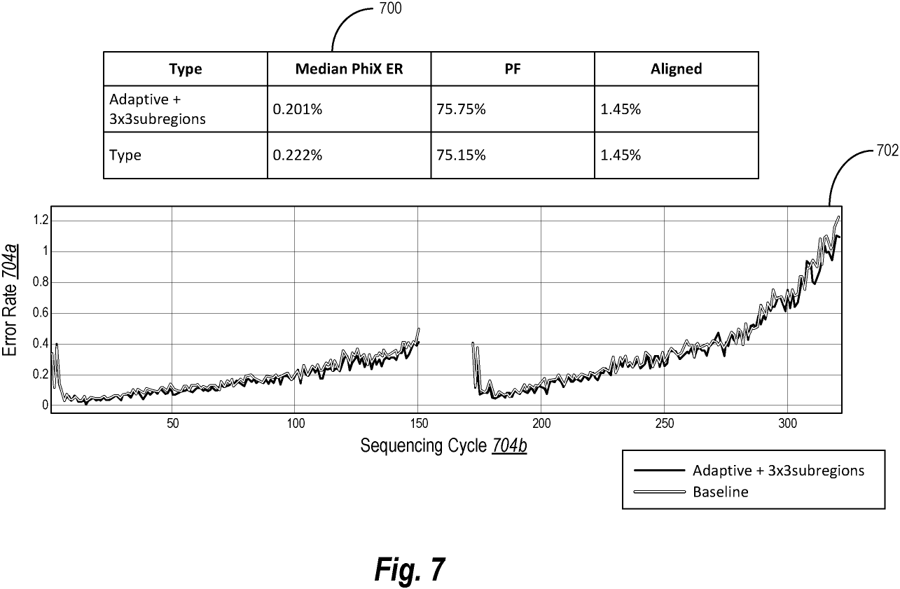

As noted below, in some embodiments, the adaptive sequencing system determines sequencing metrics for nucleobase calls of nucleotide reads. As used herein, the term “sequencing metric” refers to a quantitative measurement or score indicating a degree to which an individual nucleobase call (or a sequence of nucleobase calls) aligns, compares, or quantifies with respect to a genomic coordinate or genomic region of a reference genome, with respect to nucleobase calls from nucleotide reads, or with respect to external genomic sequencing or genomic structure. For instance, a sequencing metric includes a quantitative measurement or score indicating a degree to which (i) individual nucleobase calls align, map, or cover a genomic coordinate or reference base of a reference genome (e.g., MAPQ, depth metrics); (ii) nucleobase calls compare to reference or alternative nucleotide reads in terms of mapping, mismatch, base call quality, or other raw sequencing metrics (e.g., Q score according to a Phred algorithm, callability metrics, read-reference-mismatch metric); or (iii) genomic coordinates or regions corresponding to nucleobase calls demonstrate mappability, repetitive base call content, DNA structure, or other generalized metrics (e.g., mappability metric indicating difficulty of mapping nucleotide sequence, guanine-cytosine-content metric. As described further below with respect to FIG. 7 and other portions of this disclosure, in certain embodiments, the adaptive sequencing system determines base-call error rates as a sequencing metric by comparing nucleobase calls of a base-calling-neural network to a well-known genome, such as PhiX. When fragments of a small and well-known genome, such as PhiX, are mixed into a sequencing run, for example, the adaptive sequencing system can utilize nucleobase calls corresponding to the well-known genome and an on-sequencing device aligner to determine nucleobase error rates.

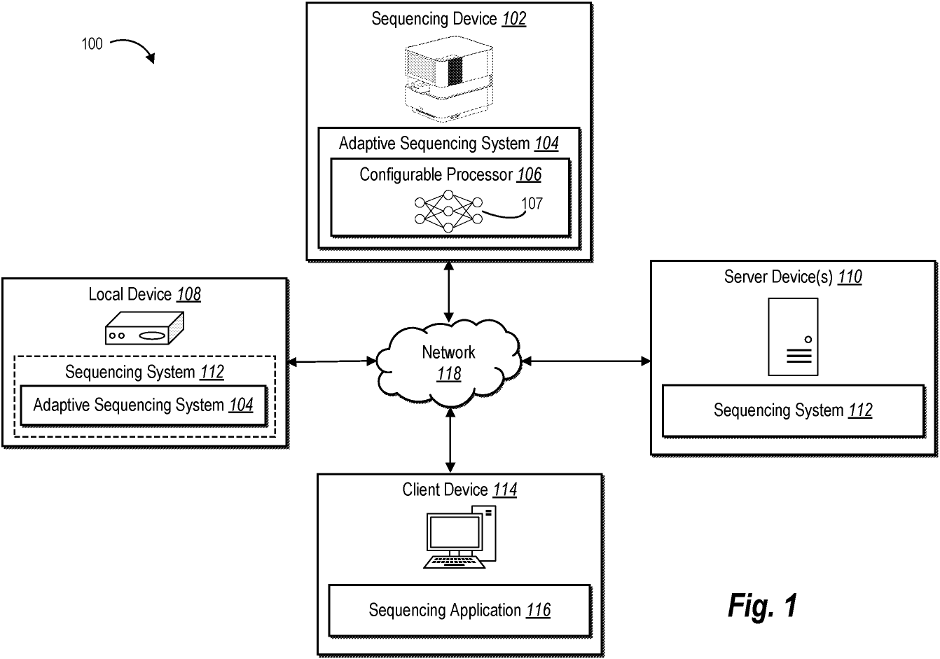

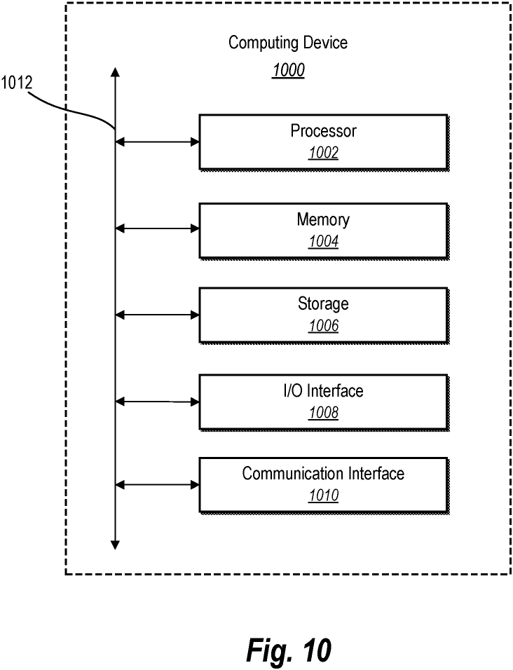

The following paragraphs describe the adaptive sequencing system with respect to illustrative figures that portray example embodiments and implementations. For example, FIG. 1 illustrates a schematic diagram of a computing system 100 in which an adaptive sequencing system 104 operates in accordance with one or more embodiments. As illustrated, the computing system 100 includes a sequencing device 102 connected to a local device 108 (e.g., a local server device), one or more server device(s) 110, and a client device 114. As shown in FIG. 1, the sequencing device 102, the local device 108, the server device(s) 110, and the client device 114 can communicate with each other via the network 118. The network 118 comprises any suitable network over which computing devices can communicate. Example networks are discussed in additional detail below with respect to FIG. 10. While FIG. 1 shows an embodiment of the adaptive sequencing system 104, this disclosure describes alternative embodiments and configurations below.

As indicated by FIG. 1, the sequencing device 102 comprises a device for sequencing a genomic sample or other nucleic-acid polymer. In some embodiments, the sequencing device 102 analyzes nucleotide fragments or oligonucleotides extracted from genomic samples to generate nucleotide-fragment reads or other data utilizing computer implemented methods and systems (described herein) either directly or indirectly on the sequencing device 102. More particularly, the sequencing device 102 receives nucleotide-sample slides (e.g., flow cells) comprising nucleotide fragments extracted from samples and then copies and determines the nucleobase sequence of such extracted nucleotide fragments. In one or more embodiments, the sequencing device 102 utilizes SBS to sequence nucleotide fragments into nucleotide-fragment reads. In addition or in the alternative to communicating across the network 118, in some embodiments, the sequencing device 102 bypasses the network 118 and communicates directly with the local device 108 or the client device 114.

As indicated above, in some embodiments, the adaptive sequencing system 104 configures a configurable processor 106, such as an ASIC, ASSP, CGRA, or FPGA, on the sequencing device 102 to implement a neural network 107, such as a base-calling-neural network, that includes a first set of layers and a second set of layers. The adaptive sequencing system 104 provides a dataset, such as a set of images of oligonucleotide clusters, to the neural network 107 and generates predicted classes, such as nucleobase calls for oligonucleotide clusters based on the set of images. Based on the nucleobase calls or other predicted classes, the adaptive sequencing system 104 subsequently uses the configurable processor 106 to modify certain network parameters for the second set of layers of the neural network 107 while maintaining certain network parameters for the first set of layers. The adaptive sequencing system 104 can further store the nucleobase calls as part of base-call data that is formatted as a BCL file and send the BCL file to the local device 108 and/or the server device(s) 110.

As further indicated by FIG. 1, the local device 108 is located at or near a same physical location of the sequencing device 102. Indeed, in some embodiments, the local device 108 and the sequencing device 102 are integrated into a same computing device. The local device 108 may run a sequencing system 112 to generate, receive, analyze, store, and transmit digital data, such as by receiving base-call data or determining variant calls based on analyzing such base-call data. As shown in FIG. 1, the sequencing device 102 may send (and the local device 108 may receive) base-call data generated during a sequencing run of the sequencing device 102. By executing software in the form of the sequencing system 112, the local device 108 may align nucleotide-fragment reads with a reference genome and determine genetic variants based on the aligned nucleotide-fragment reads. The local device 108 may also communicate with the client device 114. In particular, the local device 108 can send data to the client device 114, including a variant call file (VCF) or other information indicating nucleobase calls, sequencing metrics, error data, or other metrics.

As further indicated by FIG. 1, the server device(s) 110 are located remotely from the local device 108 and the sequencing device 102. Similar to the local device 108, in some embodiments, the server device(s) 110 include a version of the sequencing system 112. Accordingly, the server device(s) 110 may generate, receive, analyze, store, and transmit digital data, such as data for determining nucleobase calls or sequencing nucleic-acid polymers. Similarly, the sequencing device 102 may send (and the server device(s) 110 may receive) base-call data from the sequencing device 102. The server device(s) 110 may also communicate with the client device 114. In particular, the server device(s) 110 can send data to the client device 114, including VCFs or other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server.

As further illustrated and indicated in FIG. 1, the client device 114 can generate, store, receive, and send digital data. In particular, the client device 114 can receive status data from the local device 108 or receive sequencing metrics from the sequencing device 102. Furthermore, the client device 114 may communicate with the local device 108 or the server device(s) 110 to receive a VCF comprising nucleobase calls and/or other metrics, such as a base-call-quality metrics or pass-filter metrics. The client device 114 can accordingly present or display information pertaining to variant calls or other nucleobase calls within a graphical user interface to a user associated with the client device 114. For example, the client device 114 can present an active sequencing interface comprising a status summary of a sequencing run, including base-call-quality metrics and/or pass-filter metrics for the sequencing run.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 10.

As further illustrated in FIG. 1, the client device 114 includes a sequencing application 116. The sequencing application 116 may be a web application or a native application stored and executed on the client device 114 (e.g., a mobile application, desktop application). The sequencing application 116 can include instructions that (when executed) cause the client device 114 to receive data from the adaptive sequencing system 104 and present, for display at the client device 114, base-call data or data from a VCF. Furthermore, the sequencing application 116 can instruct the client device 114 to display summaries for multiple sequencing runs.

As further illustrated in FIG. 1, a version of the adaptive sequencing system 104 may be located and implemented (e.g., entirely or in part) on the local device 108. In yet other embodiments, the adaptive sequencing system 104 is implemented by one or more other components of the computing system 100, such as the server device(s) 110. In particular, the adaptive sequencing system 104 can be implemented in a variety of different ways across the sequencing device 102, the local device 108, the server device(s) 110, and the client device 114. For example, the adaptive sequencing system 104 can be downloaded from the server device(s) 110 to the adaptive sequencing system 104 and/or the local device 108 where all or part of the functionality of the adaptive sequencing system 104 is performed at each respective device within the computing system 100.

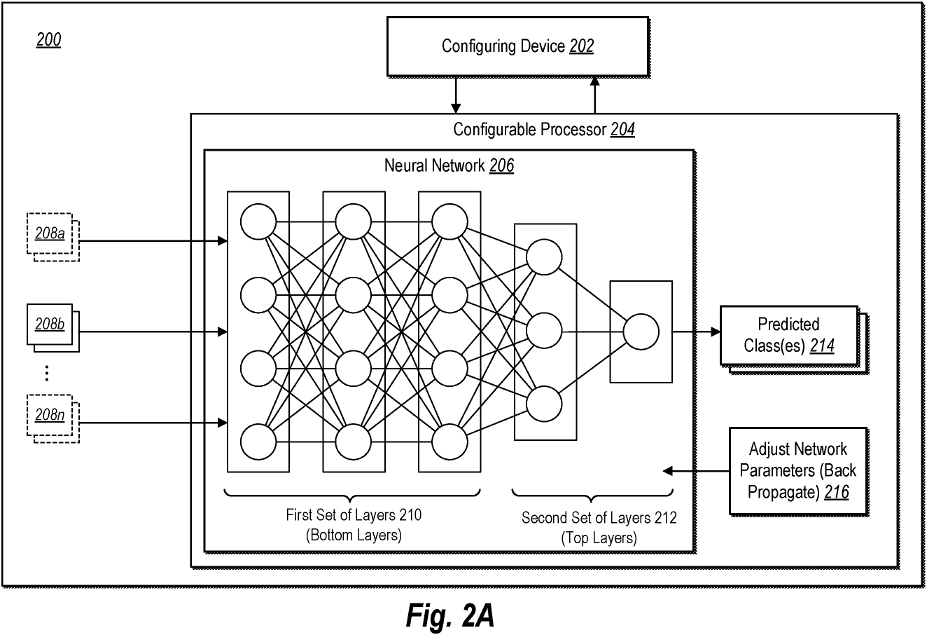

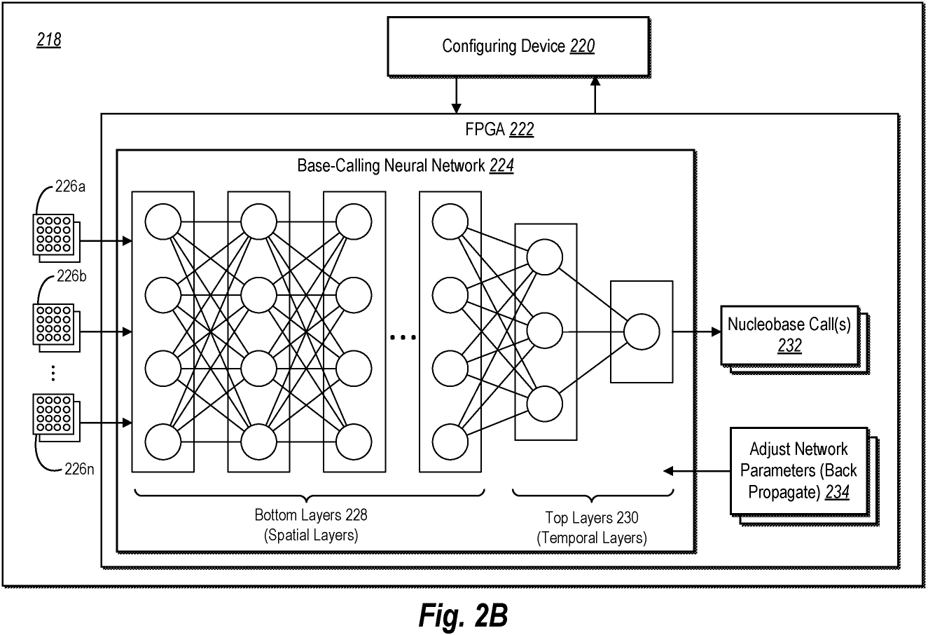

As indicated above, the adaptive sequencing system 104 can customize the training of a neural network on a local computing device by selectively modifying parameters of the neural network. In accordance with one or more embodiments, FIG. 2A illustrates an example of the adaptive sequencing system 104 (i) configuring a configurable processor to implement a neural network and (ii) training the neural network using the configurable processor by modifying certain network parameters of a subset of the neural network’s layers. FIG. 2B illustrates an example of the adaptive sequencing system 104 (i) configuring a configurable processor of a sequencing device to implement a base-calling-neural network and (ii) training the base-calling-neural network using the configurable processor by modifying certain network parameters of a subset of the base-calling-neural network’s layers.

As shown in FIG. 2A, the adaptive sequencing system 104 implements a neural network 206 on a local computing device 200. For instance, in some cases, the adaptive sequencing system 104 downloads or otherwise receives a version of the neural network 206 comprising a first set of layers 210 and a second set of layers 212 with network parameters initially trained by an external computing device. As suggested above, in one or more embodiments, the first set of layers 210 and the second set of layers 212 comprise or constitute a set of bottom layers and a set of top layers, respectively, for a CNN, RNN, LSTM, or other type of neural network. In some embodiments, a remote computing device uses a collection of GPUs and/or CPUs to train weights or other parameters of both the first set of layers 210 and the second set of layers 212 based on training data, such as training images, training videos, or training documents. After an initial version of the neural network 206 is trained, the adaptive sequencing system 104 downloads or otherwise receives data representing the initial version of the neural network 206. For instance, in some circumstances, the local computing device 200 downloads or otherwise copies a configuration file (or files), such as FPGA bit files, and network parameters corresponding to the neural network 206.

As further shown in FIG. 2A, the adaptive sequencing system 104 uses a configuring device 202 to configure a configurable processor 204 of the local computing device 200 to implement the neural network 206. The configurable processor 204 may constitute an ASIC, ASSP, CGRA, FPGA, or other configurable processor. For instance, the adaptive sequencing system 104 uses a microcontroller on a board, a boot-PROM, or an external computing device as the configuring device 202 comprising instructions to configure the configurable processor 204 to implement an initial version of the neural network 206. In some cases, the adaptive sequencing system 104 includes a program or computer-executable instructions that cause the configuring device 202 to configure or reconfigure the configurable processor 204.

After configuring the configurable processor 204, in some embodiments, the adaptive sequencing system 104 provides a dataset to the neural network 206. As depicted in FIG. 2A, for instance, the adaptive sequencing system 104 provides a dataset 208b or, in some cases, each of datasets 208a, 208b, and 208n to the neural network 206. For example, the adaptive sequencing system 104 can provide images, video, sequencing metrics, digital documents, text, or other input data as the datasets 208a-208n.

The adaptive sequencing system 104 can also input datasets corresponding to different time periods. For instance, in some cases, the adaptive sequencing system 104 provides, to the neural network 206, the dataset 208a corresponding to a subsequent time period, the dataset 208b corresponding to a target time period, and the dataset 208n corresponding to a prior time period. As explained further below, such datasets may come in the form of images. Accordingly, in certain implementations, the adaptive sequencing system 104 provides, to the neural network 206, a prior-period image corresponding to a prior time period, a target-period image corresponding to a target time period, and a subsequent-period image corresponding to a subsequent time period. Based on the prior-period image, the target-period image, and the subsequent-period image, the adaptive sequencing system 104 generates one or more predicted classes for the target time period.

Depending on the architecture of the neural network 206, therefore, the adaptive sequencing system 104 can account for time relationships through inputting and analyzing datasets corresponding to different time periods. While FIG. 2A depicts the datasets 208a, 208b, and 208n as inputs for an iteration or cycle, in some embodiments, the neural network 206 passes or processes a different number of combinations or time-period-based datasets (e.g., separate datasets for five different time periods).

Based on one or more of the datasets 208a - 208n, the adaptive sequencing system 104 uses the neural network 206 to generate predicted class(es) 214. Such predicted class(es) may come in the form of a probability or probabilities corresponding to one or more of multiple classes. For instance, in some embodiments, the neural network 206 generates facial classifications, textual classifications, object classifications, nucleobase calls, or other predicted classifications. Indeed, in some cases, the neural network 206 generates the predicted class(es) 214 for a target time period based on datasets for not only the target time period but also on datasets for prior and/or subsequent time periods.

Based on the predicted class(es) 214, the adaptive sequencing system 104 modifies network parameters 216 for a subset of layers of the neural network 206. For instance, in some embodiments, the adaptive sequencing system 104 modifies network parameters for the second set of layers 212 (e.g., top layers) and certain network parameters for a subset of the first set of layers 210 (e.g., a subset of bottom layers). For parameter adjustments, in some embodiments, the adaptive sequencing system 104 determines a gradient based on an error signal derived from the predicted class(es) 214 and, based on the determined gradient, modifies certain network parameters for a subset of layers. Alternatively, the adaptive sequencing system 104 compares the predicted class(es) 214 to ground-truth class(es) and, based on a loss or cost between the predicted class(es) 214 and the ground-truth class(es), modifies certain network parameters for a subset of layers.

As further depicted by FIG. 2A, the adaptive sequencing system 104 continues to run iterations of data through the neural network 206 and determine predicted class(es) based on additional datasets for each iteration or cycle. In some cases, the adaptive sequencing system 104 selectively modifies network parameters after or as part of selected iterations, such as every 25 or 50 iterations or cycles. Additionally or alternatively, the adaptive sequencing system 104 performs iterations or cycles until the network parameters (e.g., value or weights) of the neural network 206 do not change significantly across iterations or otherwise satisfy a convergence criteria.

As noted above, the adaptive sequencing system 104 can configure and selectively train network parameters for layers of a base-calling-neural network. In accordance with one or more embodiments, FIG. 2B depicts the adaptive sequencing system 104 (i) configuring a FPGA to implement a base-calling-neural network, (ii) generating one or more nucleobase calls for oligonucleotide clusters based on corresponding oligonucleotide-cluster images, and (iii) modifying network parameters of a subset of layers of the base-calling-neural network based on the nucleobase calls.

As shown in FIG. 2B, the adaptive sequencing system 104 implements a base-calling-neural network 224 on a sequencing device 218. For instance, in some cases, the adaptive sequencing system 104 downloads or otherwise receives a version of the base-calling-neural network 224. As initially received the version of the base-calling-neural network 224 comprises a set of bottom layers 228 and a set of top layers 230 with network parameters initially trained by an external computing device. In one or more embodiments, the set of bottom layers 228 and the set of top layers 230 comprise a set of spatial layers and a set of temporal layers, respectively, for a CNN or other type of base-calling-neural network. As part of the initial training, in some cases, a remote server and/or a remote sequencing device uses a collection of GPUs and/or CPUs to train weights or other parameters of both the set of bottom layers 228 and the set of top layers 230 based on training images of oligonucleotide clusters. After an initial version of the base-calling-neural network 224 is trained, in certain implementations, the adaptive sequencing system 104 via the sequencing device 218 or a corresponding local server device downloads or otherwise copies a configuration file (or files), such as FPGA bit files, and network parameters corresponding to the base-calling-neural network 224.

As further shown in FIG. 2B, the adaptive sequencing system 104 uses a configuring device 220 (e.g., a microcontroller on a board, a boot-PROM, or an external computing device) to configure a FPGA 222 of the sequencing device 218 to implement the base-calling-neural network 224. In some cases, the adaptive sequencing system 104 includes a program or computer-executable instructions that cause the configuring device 220 to configure the FPGA 222. For instance, the adaptive sequencing system 104 can configure a FPGA (or other configurable processor) to implement a base-calling-neural network as described by Kishore Jaganathan et al., Artificial Intelligence-Based Base Calling, U.S. Pat. Application No. 16/826,126 (filed Mar. 20, 2020) (hereinafter, Jaganathan), which is hereby incorporated by reference in its entirety.

After configuring the FPGA 222, in some embodiments, the adaptive sequencing system 104 provides one or more oligonucleotide-cluster images to the base-calling-neural network 224. As depicted in FIG. 2B, for instance, the adaptive sequencing system 104 provides, to the base-calling-neural network 224, a target-cycle image 226b of oligonucleotide clusters corresponding to a target sequencing cycle. In some cases, the adaptive sequencing system 104 provides, as inputs to the base-calling-neural network 224 for an iteration, a prior-cycle image 226a of oligonucleotide clusters corresponding to a prior sequencing cycle, the target-cycle image 226b of oligonucleotide clusters corresponding to a target sequencing cycle, and a subsequent-cycle image 226n of oligonucleotide clusters corresponding to a subsequent sequencing cycle.

In some cases, the prior-cycle image 226a, the target-cycle image 226b, and the subsequent-cycle image 226n (i) each constitute an image patch extracted from a larger image captured by a camera (e.g.., CCD camera), (ii) each center around a target cluster of oligonucleotides, and (iii) together serve as a basis for determining nucleobase calls for the target sequencing cycle. Further, in some embodiments, the base-calling-neural network 224 passes or processes a different number of sequencing cycle images (e.g., separate images of the same oligonucleotide clusters for five different sequencing cycles).

Based on one or more of the prior-cycle image 226a, the target-cycle image 226b, and the subsequent-cycle image 226n, the adaptive sequencing system 104 uses the base-calling-neural network 224 to generate nucleobase call(s) 232 for the target sequencing cycle. Such nucleobase calls may come in the form of a base-call probability or base-call probabilities that a cluster of oligonucleotides incorporated a particular nucleobase class (e.g., A, G, C, T) and emitted a light signal corresponding to the particular incorporated nucleobase class, as depicted in the target-cycle image 226b. In particular, in some embodiments, the base-calling-neural network 224 generates the nucleobase call(s) 232 as base-call probabilities that respective clusters of oligonucleotides incorporated particular nucleobase classes (e.g., A, G, C, T) and emitted respective light signals corresponding to the particular incorporated nucleobase classes, as depicted in the target-cycle image 226b. Accordingly, for a given target sequencing cycle and a given target-cycle image, the base-calling-neural network 224 can generate individual nucleobase calls corresponding to individual clusters of oligonucleotides, such as a first nucleobase call corresponding to a first cluster of oligonucleotides depicted in the target-cycle image 226b and a second nucleobase call corresponding to a second cluster of oligonucleotides depicted in the target-cycle image 226b.

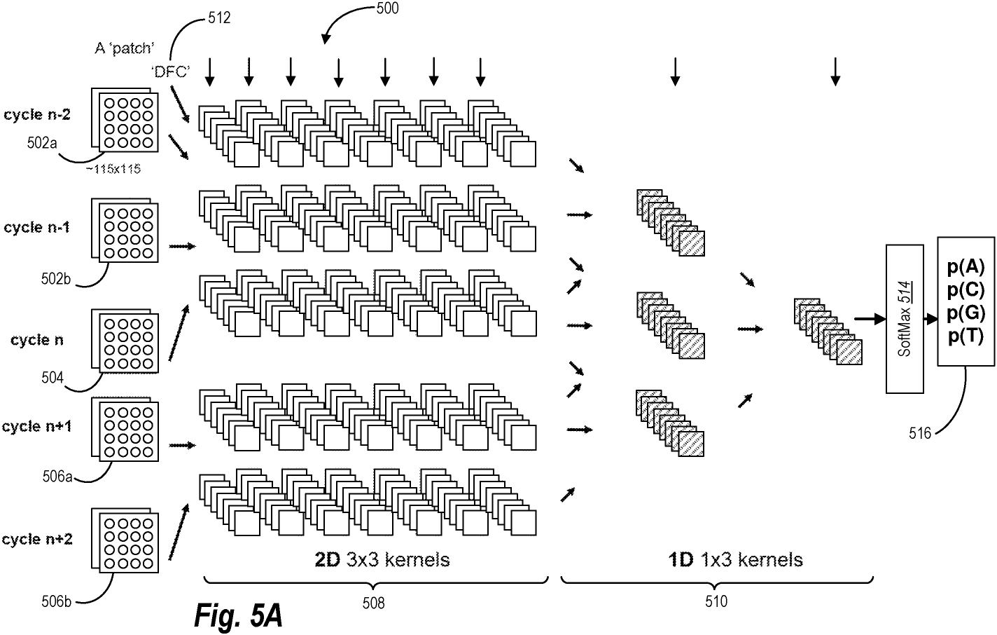

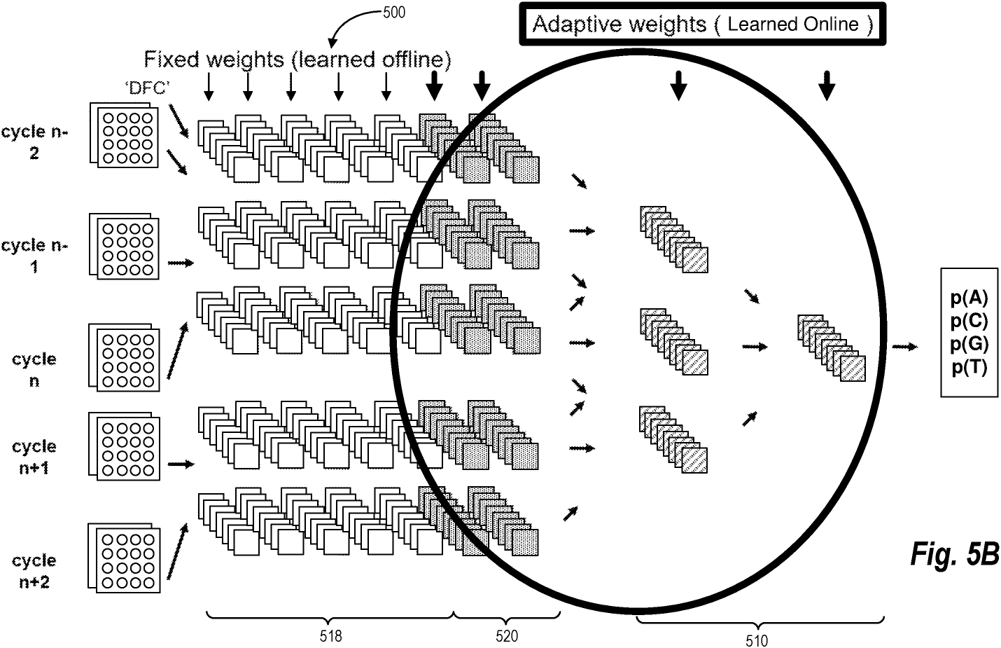

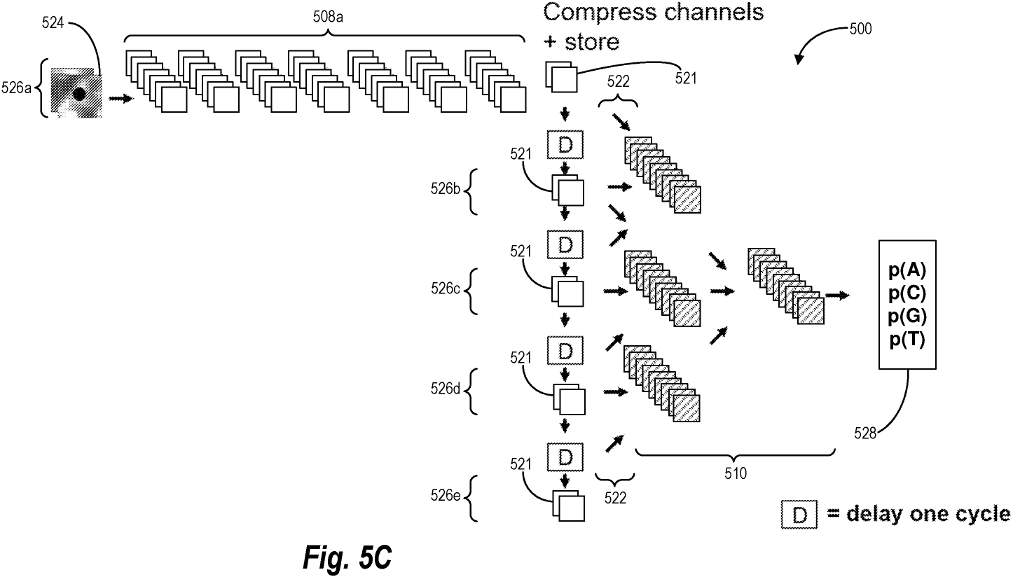

Based on the nucleobase call(s) 232 for the target sequencing cycle, the adaptive sequencing system 104 modifies network parameters 234 for a subset of layers of the base-calling-neural network 224. For instance, in some embodiments, the adaptive sequencing system 104 modifies high-resolution weights or other network parameters for the set of top layers 230 (e.g., temporal layers) and certain network parameters for a subset of the set of bottom layers 228 (e.g., a subset of spatial layers). This disclosure further describes examples of adjusting a subset of layers with respect to FIG. 5B.

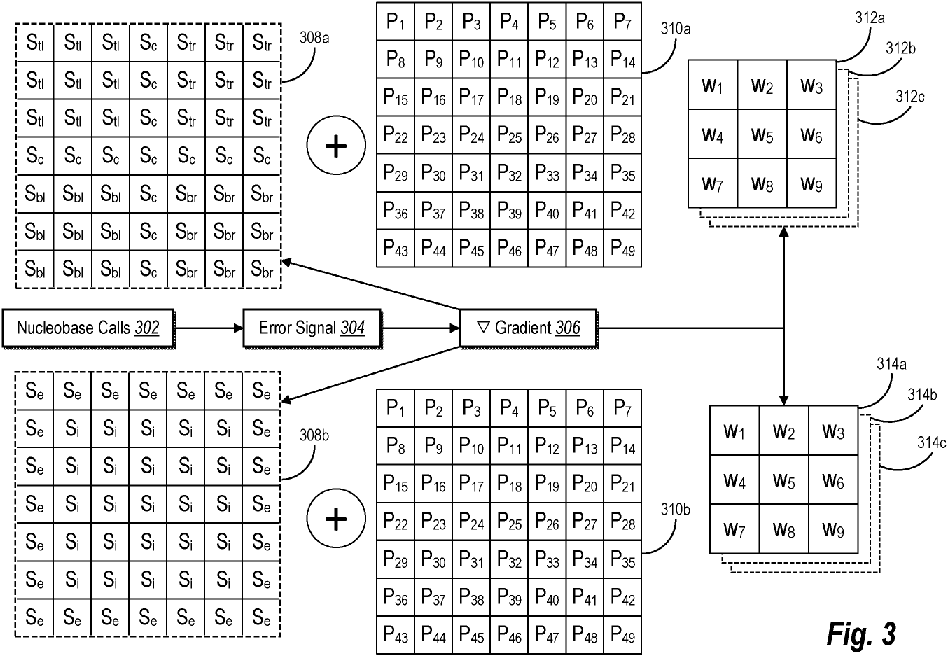

For parameter adjustments, in some embodiments, the adaptive sequencing system 104 determines a gradient based on an error signal derived from the nucleobase call(s) 232 and, based on the determined gradient, modifies certain network parameters for a subset of layers. This disclosure further describes examples of gradients below with respect to FIG. 3. Alternatively, the adaptive sequencing system 104 compares the nucleobase call(s) 232 to ground-truth nucleobase call(s) and, based on a loss or cost between the nucleobase call(s) 232 and the ground-truth nucleobase call(s), modifies certain network parameters for a subset of layers.

As further depicted by FIG. 2B, the adaptive sequencing system 104 continues to run iterations of oligonucleotide-cluster images through the base-calling-neural network 224 and determine nucleobase call(s) based on additional oligonucleotide-cluster images for each iteration or cycle. In some cases, the adaptive sequencing system 104 selectively modifies network parameters after (or as part of) selected iterations, such as every 25 or 50 sequencing cycles. Additionally or alternatively, the adaptive sequencing system 104 performs iterations or cycles until the network parameters (e.g., value or weights) of the base-calling-neural network 224 do not change significantly across iterations or otherwise satisfy a convergence criteria.

As noted above, in some embodiments, the adaptive sequencing system 104 determines an error or loss based on predicted class(es), determines a gradient for adjusting one or more network parameters, and modifies network parameters corresponding to particular images (or particular image regions) based on the gradient. In accordance with one or more embodiments, FIG. 3 illustrates the adaptive sequencing system 104 determining an error signal based on nucleobase calls; determining, from the error signal, a gradient with a fixed-point range for adjusting one or more network parameters; and modifying one or more network parameters corresponding to particular images or particular image regions based on the fixed-point-range gradient. While the following description of FIG. 3 focuses on a base-calling-neural network, oligonucleotide-cluster images, a sequencing device, and nucleobase calls, the adaptive sequencing system 104 can perform the same type of actions described below with respect to another neural network, input images, computing device, and predicted class(es).

As shown in FIG. 3, for instance, the adaptive sequencing system 104 determines nucleobase call(s) 302 utilizing a base-calling-neural network and subsequently determines an error signal 304 based on the nucleobase call(s) 302. In some cases, the adaptive sequencing system 104 derives the error signal 304 from the nucleobase call(s) 302 by assuming or utilizing a nucleobase call for a target sequencing cycle as a correct nucleobase call. For example, as noted above, in certain implementations, the base-calling-neural network generates base-call probabilities that particular nucleobase types (e.g., A, C, G, T) constitute a correct nucleobase call based in part on an oligonucleotide-cluster image for a target cluster and a target sequencing cycle. As described further below with respect to FIG. 5A, for instance, the base-calling-neural network can include a softmax layer that generates a vector or other output comprising a base-call probability for each of A, C, G, and T (e.g., in a vector [0.75, 0.15, 0.8, 0.2]). By assuming or utilizing a highest base-call probability as a correct nucleobase call and representing the correct nucleobase call with a value (e.g., 1), the adaptive sequencing system 104 can determine a value difference between the highest base-call probability (e.g., 0.75) and the value representing the presumably correct nucleobase call (e.g., 1 from a vector [1, 0, 0, 0]). Accordingly, in some embodiments, such a value difference represents the error signal 304.

To illustrate, in some embodiments, the adaptive sequencing system 104 determines a loss based on a comparison between one or more base-call probabilities (e.g., 0.55 from a vector [0.55, 0.30, 0.10, 0.05]) and a value representing the presumably correct nucleobase call (e.g., 1 from a vector [1, 0, 0, 0]) using a loss function. In particular, the adaptive sequencing system 104 can use a cross-entropy loss function to determine a cross-entropy loss between the base-call probabilities (e.g., [0.55, 0.30, 0.10, 0.05]) determined by the base-calling-neural network and value(s) representing the presumably correct nucleobase call (e.g., [1, 0, 0, 0]. Accordingly, in some embodiments, the cross-entropy loss value for a target sequencing cycle and/or a target cluster of oligonucleotides represents the error signal 304.

As suggested above, in some cases, the adaptive sequencing system 104 can selectively determine the error signal 304 (and a gradient for tuning network parameters) after multiple sequencing cycles or after or during selected sequencing cycles. For instance, in certain implementations, the adaptive sequencing system 104 determines a value difference or loss for a target sequencing cycle in between (or after) multiple sequencing cycles for which no value difference or loss was determined-thereby adjusting network parameters after selected sequencing cycles.

Additionally, or alternatively, the adaptive sequencing system 104 uses a value difference or loss to determine a gradient for adjusting network parameters when such a value difference or loss (or base-call probability) satisfies a threshold value, but not when above the threshold value. As an example of such a threshold value, in some embodiments, the adaptive sequencing system 104 uses as a threshold difference of + or - 0.50, + or - 0.60, or + or - 0.75-or other difference value-between a highest base-call probability and a value of 1 representing the presumably correct nucleobase call. When the value difference or a highest base-call probability (e.g., 0.30) does not satisfy the threshold value (e.g., + or - 0.70), in some cases, the adaptive sequencing system 104 forgoes adjusting network parameters for the corresponding sequencing cycle. By clipping or ignoring value differences or losses that exceed such a threshold error, in some embodiments, the adaptive sequencing system 104 facilitates convergence upon turned network parameters. Such an upper threshold value or upper threshold loss is compatible with a gradient with a fixed-point range by limiting an upper range on the gradient, as described further below.

In some embodiments, the adaptive sequencing system 104 does not use a lower threshold value for a difference or loss (or base-call probability) as a basis for adjusting or updating certain network parameters. Indeed, in some cases, the adaptive sequencing system 104 relies on relatively small difference or loss values in iterations to incrementally adjust network parameters until a point of convergence. In addition to relying on a relatively small value difference or loss for a single iteration, in some embodiments, the adaptive sequencing system 104 accumulates a subset of value differences or losses across multiple iterations and determines a gradient for adjusting network parameters based on the accumulated subset of value differences or losses, such as by averaging the subset of value differences or losses.

In contrast to using a presumably correct nucleobase call to determine the error signal 304, in some embodiments, the adaptive sequencing system 104 compares the nucleobase call(s) 302 to ground-truth nucleobase call(s) to determine the error signal 304. Such ground-truth nucleobase call(s) may be used when the nucleobase call(s) predict nucleobase types for a known genome (e.g., PhiX or other reference genome). In certain sequencing runs, for instance, the adaptive sequencing system 104 performs sequencing cycles for oligonucleotide clusters comprising (i) oligonucleotides of a known nucleotide sequence (e.g., oligonucleotides from a reference genome) and (ii) other oligonucleotide clusters comprising oligonucleotides from one or more sample genomes with an unknown nucleotide sequence. In such cases, the adaptive sequencing system 104 can selectively compare nucleobase calls for oligonucleotide clusters with a known nucleotide sequence to particular nucleobase types in the known nucleotide sequence. For example, in some embodiments, the adaptive sequencing system 104 uses a cross-entropy loss function to determine a loss between a value for the ground-truth nucleobase call (e.g., 1) and a base-call probability for the predicted nucleobase call (e.g., 0.55). In the alternative to determining a loss using a cross-entropy-loss function, in some embodiments, the adaptive sequencing system 104 uses binary cross-entropy loss, mean-squared error loss, L1 loss, L2 loss, smooth L1 loss, Huber loss, or other suitable loss function, as further described by Jaganathan.

Based on the error signal 304, the adaptive sequencing system 104 determines a gradient 306 for adjusting network parameters. While FIG. 3 depicts a single gradient, in some embodiments, the adaptive sequencing system 104 accumulates or determines a gradient for each node of the subset of layers with network parameters to be modified. In some embodiments, for instance, the adaptive sequencing system 104 uses a form of Gradient Descent (GD) to determine the gradient 306 and adjust parameters. To perform a form of GD, for instance, the adaptive sequencing system 104 identifies or selects a starting value for a network parameter (e.g., a weight for a selected layer of a base-calling-neural network). Because an initial version of a base-calling-neural network can be trained by another computing device, in some embodiments, the adaptive sequencing system 104 identifies a network parameter corresponding to a previously trained layer of a base-calling-neural network selected for modification during a sequencing run (e.g., a top layer, a temporal layer).

The adaptive sequencing system 104 further determines the gradient 306 of a loss curve from the starting value of the network parameter (e.g., gradient as a derivative of the loss curve or partial derivative of the loss curve). In some embodiments, for instance, the adaptive sequencing system 104 determines the gradient 306 as a vector of partial derivatives of the loss curve. To reduce a loss for a subsequent iteration and move toward a minimum loss, the adaptive sequencing system 104 determines a value for the relevant network parameter along a negative gradient (e.g., by adding a fraction of the gradient’s magnitude to the starting value). By iteratively adjusting a value for the relevant network parameter over multiple iterations or sequencing cycles, the adaptive sequencing system 104 converges toward a minimum loss.

As suggested above, in some embodiments, the adaptive sequencing system 104 determines the gradient 306 with a fixed-point range rather than a floating-point range. While existing sequencing systems commonly use floating-point ranges that include an indeterminate or variable number of values in a vector representing a gradient, existing sequencing systems often consume more computer processing to determine the values of such a floating-point-range gradient. If a configurable processor on a computing device, such as an FPGA of a sequencing device, were used for training a deep neural network, the computer processing for a floating-point range would tax the configurable processor and consume considerable time. To avoid such taxing computer processing and expedite training during a sequencing run, the adaptive sequencing system 104 determines the gradient 306 with a fixed-point range rather than a floating-point range. For instance, the adaptive sequencing system 104 determines the gradient 306 as a vector comprising values (e.g., partial derivatives) for a fixed number of positions within the vector.

By using such a fixed-point-range gradient, the adaptive sequencing system 104 conserves memory, limits parameter storage, conserves inference time, and reduces processing consumption and power used on a sequencing device. Because a fixed-point-range gradient can conserve memory and inference time for adjusting network parameters, in some embodiments, the adaptive sequencing system 104 can also increase a model size for a base-calling-neural network with additional available memory and speed up logic with less (or no) time consumed on a floating-point range. Further, by using such a fixed-point-range gradient, the adaptive sequencing system 104 increases a number of available data paths by requiring less digital logic and fewer registers or gates (e.g., in FPGA).

To further facilitate and expedite adjusting network parameters for a base-calling-neural network on a sequencing device, in some embodiments, the adaptive sequencing system 104 uses a form of Stochastic Gradient Descent (SGD) to adjust or train a subset of a neural network’s layers. While the adaptive sequencing system 104 can determine an error signal based on some (or all) nucleobase calls of a sequencing cycle, in certain implementations, the adaptive sequencing system 104 determines (i) error signals for a sequencing cycle based on a subset of nucleobase calls from a subset of oligonucleotide clusters in a minibatch SGD or (ii) an error signal for a sequencing cycle based on a nucleobase call from an oligonucleotide cluster in a single batch for SGD. Such nucleobase calls can be chosen at random by the adaptive sequencing system 104 for a sequencing cycle and for adjusting a subset of neural network layers. Because a given sequencing cycle or a given sequencing run can include hundreds of thousands, millions, or more nucleobase calls from a sequencing device, the adaptive sequencing system 104 can efficiently use a form of SGD to determine adjusted network parameters.

In addition or in the alternative to SGD as a learning optimizer, in some embodiments, the adaptive sequencing system 104 uses an Adam optimizer to update a learning rate at different iterations, such as by updating a learning rate for each weight. The adaptive sequencing system 104 can also use other suitable learning optimizers, such as adaptive learning rates, batching, and averaging losses across iterations.

Based on the gradient 306, in some embodiments, the adaptive sequencing system 104 modifies network parameters for a subset of layers within a base-calling-neural network. In some cases, the network parameters can be specific to a region or subregion of an image, such as by modifying scaling values for image-region-specific (or image-subregion-specific) scaling channels or weights for image-region-specific (or image-subregion-specific) filters. In some cases, a subregion of an image (or image patch) represents a subtile from a tile of a nucleotide-sample slide. In particular, a subregion of an image patch represents one of 3×3 subregions, which are described further below with respect to FIG. 7.

As shown in FIG. 3, in certain implementations, the adaptive sequencing system 104 (i) identifies one or more of a scaling channel 308a or 308b from a layer of a base-calling-neural network or a filter 312a or 314a from a layer of a base-calling-neural network for adjustment and (ii) adjusts one or more of scaling values for the scaling channel 308a or 308b or weights for the filter 312a or 314a. In some cases, the scaling channels 308a and 308b and the filters 312a and 314a represent scaling channels and filters as described by Artificial Intelligence-Based Base Calling, U.S. Application No. 16/826,126 (filed Mar. 20, 2020), which is hereby incorporated by reference in its entirety.

As further shown in FIG. 3, image maps 310a and 310b represent a numerical representation of (i) pixel values of an image or an image patch or (ii) pixel values of an image or image patch from a larger oligonucleotide-cluster image, where the pixel values have been combined, consolidated, averaged, or otherwise modified. When applied to the image map 310a or 310b by a base-calling-neural network, for instance, the scaling values within the scaling channel 308a or 308b modify the values within the image map 310a or 310b, respectively. When applied to the image map 310a or 310b in a sliding window (e.g., 9×9) by the base-calling-neural network, the weights within the filter 312a or 314a modify the values within the image map 310a or 310b, respectively, to generate a new image map (e.g., an image map of smaller dimension).

As further shown in FIG. 3, the scaling channels 308a and 308b include scaling values specific to a region of an image or a subregion of an image corresponding to the image maps 310a and 310b, respectively. For instance, the scaling channel 308a comprises scaling values labeled as “stl,” str,” “sc,” “sbl,” and “sbr” representing scaling values for a top-left region, a top-right region, a center region, a bottom-left region, and a bottom-right region of an image or the image map 310a. In some cases, for instance, the top-left region, top-right region, center region, bottom-left region, and bottom-right region of the image or the image map 310a include varying light signals or varying focus captured by a camera or, additionally or alternatively, correspond to different oligonucleotide clusters depicted by the image. By adjusting one or more of top-left scaling values, top-right scaling values, center scaling values, bottom-left scaling values, or bottom-right scaling values within the scaling channel 308a based on the gradient 306, the adaptive sequencing system 104 adjusts scaling values for the top-left region, top-right region, center region, bottom-left region, or bottom-right region, respectively, of an image or the image map 310a.

As another example, in certain embodiments, the scaling channel 308b comprises scaling values labeled as “se” and “si” representing scaling values for an edge region or an internal region of an image or the image map 310b, respectively. In some cases, for instance, the edge region or internal region of the image or the image map 310a include varying light signals or varying focus captured by a camera or, additionally or alternatively, correspond to different oligonucleotide clusters depicted by the image. By adjusting one or more of edge scaling values and internal scaling values within the scaling channel 308b based on the gradient 306, the adaptive sequencing system 104 adjusts scaling values for the edge region or internal region, respectively, of the image or the image map 310a.

As further shown in FIG. 3, the filters 312a and 314a include weights specific to different types of images represented by the image maps 310a and 310b, respectively. For instance, the image map 310a may represent an image patch extracted or removed from one region (e.g., top-left region) of a larger oligonucleotide-cluster image and the image map 310b may represent another image patch extracted or removed from another region (e.g., center region) of the larger oligonucleotide-cluster image. Accordingly, in some embodiments, the weights w₁ - w₉ within the filter 312a are specific to one region of a larger image and the weights w₁ - w₉ within the filter 314a are specific to another region of the larger image. By adjusting one or more of weights within the filter 312a or 314a based on the gradient 306, the adaptive sequencing system 104 adjusts weights for different regions of an image represented by the image map 310a or 310b, respectively.

Additionally or alternatively, as an optional feature, the adaptive sequencing system 104 uses multiple image-region-specific filters on a single image map in a layer of a base-calling-neural network. As further shown in FIG. 3, in some embodiments, the adaptive sequencing system 104 optionally adjusts weights within filters corresponding to different regions or subregions within an image or image map. As depicted in FIG. 3, a base-calling-neural network may include filters 312a, 312b, and 312c that the adaptive sequencing system 104 applies to different regions of the image map 310a. For instance, in some embodiments, the adaptive sequencing system 104 applies the filters 312a, 312b, and 312c as a sliding window along to a top row, middle row, and bottom row of the image map 310a. Similarly, the base-calling-neural network may include filters 314a, 314b, and 314c that the adaptive sequencing system 104 applies to different regions of the image map 310b. For instance, in some embodiments, the adaptive sequencing system 104 applies the filters 314a, 314b, and 314c as a sliding window along to a top row, middle row, and bottom row of the image map 310b. The filters 312a, 312b, and 312c can include the same weights as (or different weights from) the filters 314a, 314b, and 314c, respectively. Further, the top, middle, and bottom rows of the image map 310a and the image map 310b represent different regions of corresponding images. By selectively modifying the weights of one or more of the filters 312a, 312b, or 312c, or the filters 314a, 314b, or 314c, the adaptive sequencing system 104 modifies subsets of weights corresponding to different regions or subregions of images.

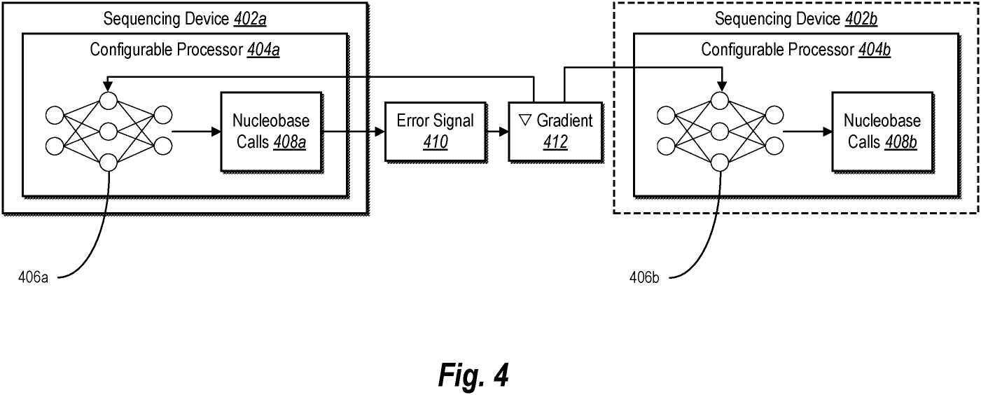

In addition to using predicted class(es) and a gradient to train layers of one neural network, in some embodiments, the adaptive sequencing system 104 can determine a gradient based on an error signal derived from predicted class(es) of a one neural network to adjust a subset of network parameters for a subset of layers within multiple neural networks. In accordance with one or more embodiments, FIG. 4 illustrates the adaptive sequencing system 104 adjusting a subset of layers for multiple base-calling-neural networks using configurable processors-based on a gradient or loss determined from predicted classes of a single base-calling-neural network. While the following description of FIG. 4 focuses on a base-calling-neural network, a sequencing device, and nucleobase calls, the adaptive sequencing system 104 can perform the same type of actions described below with respect to another neural network, computing devices, and predicted class(es).

As shown in FIG. 4, in some embodiments, an instance or version of the adaptive sequencing system 104 configures a configurable processor 404a to implement a base-calling-neural network 406a on a sequencing device 402a. Similarly, an instance or version of the adaptive sequencing system 104 configures a configurable processor 404b to implement a base-calling-neural network 406b on a sequencing device 402b. For instance, in some cases, the adaptive sequencing system 104 (i) receives or downloads an initial version of a base-calling-neural network comprising layers initially trained by another computing device (e.g., remote servers) and (ii) configures both the configurable processor 404a and the configurable processor 404b to implement the initial version of the base-calling-neural network as the base-calling-neural network 406a and the base-calling-neural network 406b, respectively. As indicated by FIG. 4, the sequencing device 402b is optional. In some embodiments, the adaptive sequencing system 104 configures and modifies parameters for the base-calling-neural network 406a and the base-calling-neural network 406b on multiple FPGAs (or other configurable processors) on a single sequencing device.

After configuring the configurable processor 404a, consistent with the disclosure above, the adaptive sequencing system 104 determines nucleobase calls 408a based on oligonucleotide-cluster images, determines an error signal 410 based on the nucleobase calls 408a, and determines a gradient 412 based on the error signal 410. The adaptive sequencing system 104 can subsequently use the gradient 412 as the basis for determining modified network parameters for one or more additional base-calling-neural networks. Based on the gradient 412, for instance, the adaptive sequencing system 104 (i) modifies network parameters of a subset of layers of the base-calling-neural network 406a and (ii) modifies network parameters of a subset of layers of the base-calling-neural network 406b. For a given sequencing cycle or after a sequencing run, for instance, the adaptive sequencing system 104 can modify network parameters of a subset of layers of both the base-calling-neural network 406a and 406b. By using the base-calling-neural network 406b with now modified network parameters, an iteration or version of the adaptive sequencing system 104 determines nucleobase calls 408b based on oligonucleotide-cluster images.

While FIG. 4 depicts the adaptive sequencing system 104 adjusting a subset of layers for base-calling-neural networks 406a and 406b based on the gradient 412, the adaptive sequencing system 104 can likewise use another gradient to cross train base-calling-neural networks. For instance, in some embodiments, the adaptive sequencing system 104 uses the base-calling-neural network 406b to determine the nucleobase calls 408b based on oligonucleotide-cluster images, determines an additional error signal based on the nucleobase calls 408b, and determines an additional gradient based on the additional error signal. Based on the additional gradient, the adaptive sequencing system 104 adjusts a subset of layers for both the base-calling-neural networks 406a and 406b.

As suggested above, the adaptive sequencing system 104 can implement a base-calling-neural network using a configurable processor of a sequencing device and selectively train a subset of layers of the base-calling-neural network-during or after a sequencing run of a sequencing device. In accordance with one or more embodiments, FIGS. 5A-5C illustrate the adaptive sequencing system 104 implementing and training a base-calling-neural network during a sequencing run. In particular, FIG. 5A depicts the adaptive sequencing system 104 implementing an example architecture of a base-calling-neural network during sequencing cycles of a sequencing run. FIG. 5B depicts the adaptive sequencing system 104 modifying certain network parameters for a subset of layers within the base-calling-neural network. FIG. 5C depicts the adaptive sequencing system 104 using the base-calling-neural network to determine nucleobase calls for a target sequencing cycle based on intermediate values corresponding to a prior or subsequent sequencing cycle and generating additional nucleobase calls for the prior or subsequent sequencing cycle based on the intermediate values. While the following description of FIG. 5A focuses on a base-calling-neural network, sequencing device, sequencing cycles, and nucleobase calls, the adaptive sequencing system 104 can perform the same type of actions described below with respect to another neural network, computing device, time periods, and predicted class(es).

As shown in FIG. 5A, for instance, the adaptive sequencing system 104 implements a base-calling-neural network 500 on a sequencing device. In some embodiments, the adaptive sequencing system 104 uses a CNN as the base-calling-neural network 500 comprising at least a set of spatial layers 508, a set of temporal layers 510, and a softmax layer 514. As part of implementing the base-calling-neural network 500, in some cases, the adaptive sequencing system 104 uses the set of spatial layers 508 of the base-calling-neural network 500 to determine (and distinguish between) values representing particular light signals from oligonucleotide clusters depicted in spatial proximity within one or more images. By contrast, the adaptive sequencing system 104 uses the set of temporal layers 510 of the base-calling-neural network 500 to determine (and distinguish between) values representing light signals from the oligonucleotide clusters across different sequencing cycles. Based on feature maps output by the set of temporal layers 510, the adaptive sequencing system 104 uses the softmax layer 514 of the base-calling-neural network 500 to generate base-call probabilities.

For a target sequencing cycle, for instance, the adaptive sequencing system 104 inputs a set of images of oligonucleotide clusters associated with the target sequencing cycle. For instance, oligonucleotide-cluster images for a same subsection of a nucleotide-sample slide within a threshold number of sequencing cycles (e.g., one or two sequencing cycles) of a target sequencing cycle may be input into the base-calling-neural network 500. As shown in FIG. 5A, the adaptive sequencing system 104 inputs (i) a prior-cycle image 502a of oligonucleotide clusters for a first prior sequencing cycle before the target sequencing cycle, (ii) a prior-cycle image 502b of the oligonucleotide clusters for a second prior sequencing cycle, (iii) a target-cycle image 504 of the oligonucleotide clusters for the target sequencing cycle, (iv) a subsequent-cycle image 506a of the oligonucleotide clusters for a first subsequent sequencing cycle after the target sequencing cycle, and (v) a subsequent-cycle image 506b of the oligonucleotide clusters for a second subsequent sequencing cycle. As the names suggest, the first prior sequencing cycle precedes the second prior sequencing cycle in a sequencing run, and the first subsequent sequencing cycle precedes the second subsequent cycle in the sequencing run.

As suggested by FIG. 5A, in some embodiments, the prior-cycle images 502a and 502b, the target-cycle image 504, and the subsequent-cycle images 506a and 506b each represent an image patch extracted or removed from a larger image of oligonucleotides. For instance, each of the prior-cycle images 502a and 502b, the target-cycle image 504, and the subsequent-cycle images 506a and 506b represent an image patch of approximately 115×115 pixels. Because clusters of oligonucleotides are distributed across a nucleotide-sample slide, some pixels in a particular image patch directly represent light emitted from an oligonucleotide cluster while other pixels in the particular image patch do not directly represent such light. For instance, in some cases, an image comprises an average of one out of ten pixels that directly represent light emitted from an oligonucleotide cluster.

Because the light emitted by a fluorescently tagged oligonucleotide cluster and captured by an image can vary in intensity or dimness, in some embodiments, the adaptive sequencing system 104 also inputs state information concerning image data into the base-calling-neural network 500. As shown in FIG. 5A, for instance, in some embodiments, the adaptive sequencing system 104 inputs distance from center (DFC) data 512 corresponding to each image or image patch into the base-calling-neural network 500. In some cases, the DFC data indicates a distance a particular cluster is from a pixel center in an image. By inputting the DFC data 512, layers of the base-calling-neural network 500 can correct or normalize a brightness of light emitted by fluorescent tags associated with nucleobases incorporated into growing oligonucleotides of a given cluster. The base-calling-neural network 500, therefore, can compensate for an overly dim or overly bright light from an oligonucleotide cluster within an image relative to another light from another oligonucleotide cluster.

After extracting values from the prior-cycle images 502a and 502b, the target-cycle image 504, and the subsequent-cycle images 506a and 506b, the adaptive sequencing system 104 inputs feature maps representing such values into the set of spatial layers 508 as part of a forward path. As shown in FIG. 5A, the set of spatial layers 508 comprise various convolutional kernels for channels that determine (and distinguish between) values representing particular light signals from oligonucleotide clusters depicted in spatial proximity within an image. Such convolutional kernels may be applied as filters to feature maps from input images. In particular, the set of spatial layers 508 include five parallel paths, where one path passes or processes an initial feature map of a corresponding oligonucleotide-cluster image (e.g., the prior-cycle image 502a). In a given path of the set of spatial layers 508, the filters include two-dimensional 3×3 convolutional kernels that the base-calling-neural network 500 applies to feature maps representing input image patches. In some embodiments, the weights of each five parallel paths are the same and can be modified together during training. Accordingly, for efficient computing on an FPGA or other configurable processor, the base-calling-neural network 500 comprises shared weights across five parallel paths and relatively small convolutions. Indeed, in some cases, the set of spatial layers 508 include spatial convolution layers as described by Jaganathan.

By applying filters comprising two-dimensional convolutional kernels across pixels of each input image patch-such as the prior-cycle image 502a, the target-cycle image 504, or the subsequent-cycle image 506a-the base-calling-neural network 500 can equalize a point spread function (PSF) and correct for values representing crosstalk among different oligonucleotide clusters captured by an image patch. Accordingly, the filters of the set of spatial layers 508 efficiently filter out noise from light signals of spatially proximate oligonucleotide clusters from a target light signal of a target oligonucleotide cluster. In contrast to a neural network comprising 64 filters that would pass feature maps for three input image patches for a target sequencing cycle, the set of spatial layers 508 depicted in FIG. 5A include 14 filters in each parallel path, thereby reducing computational processing in comparison to a 64-filter variation by approximately one twelfth.

After passing image data through the set of spatial layers 508, the set of spatial layers 508 output feature maps (e.g., 14 feature maps) that feed into the set of temporal layers 510 as part of a forward path. As shown in FIG. 5A, the set of spatial layers 508 comprise various convolutional kernels for channels that determine (and distinguish between) values representing light signals from the oligonucleotide clusters across different sequencing cycles. Such convolutional kernels may be applied as filters to feature maps from input images. In particular, the set of spatial layers 508 include a layer with three parallel paths, where each path passes or processes a corresponding feature map derived from one or more oligonucleotide-cluster images (e.g., derived from the prior-cycle image 502a and the prior-cycle image 502b). As shown as a last layer, the set of spatial layers 508 further include a layer to which the three previous layers output a feature map. In each layer of the set of spatial layers 508, the filters include one-dimensional 1×3 convolutional kernels that the base-calling-neural network 500 applies to feature maps output by the set of spatial layers 508. Indeed, in some cases, the set of temporal layers 510 include temporal convolution layers as described by Jaganathan.

By applying filters comprising one-dimensional convolutional kernels across imaging data representing different sequencing cycles-such as image patches for two prior sequencing cycles, a target sequencing cycle, and two subsequent cycles-the base-calling-neural network 500 can equalize phasing and pre-phasing effects among different oligonucleotide clusters captured by image patches. The filters of the set of temporal layers 510 accordingly filter out light signals from previous or subsequent sequencing cycles for oligonucleotide clusters to distinguish values corresponding to a target sequencing cycle.

As indicated by FIG. 5A, the set of temporal layers 510 output a feature map upon which the base-calling-neural network 500 determines nucleobase calls. In some embodiments, for instance, the base-calling-neural network 500 passes an output feature map through the softmax layer 514. Based on the output feature map, the softmax layer 514 determines base-call probabilities 516 for a target cluster of oligonucleotides and the target sequencing cycle. For instance, in some cases, the softmax layer 514 outputs a first base-call probability for an adenine nucleobase call (e.g., 0.70 for A), a second base-call probability for a cytosine nucleobase call (e.g., 0.10 for C), a third base-call probability for a guanine nucleobase call (e.g., 0.10 for G), and a fourth base-call probability for a thymine nucleobase call (e.g., 0.10 for T). In some cases, the adaptive sequencing system 104 selects a highest base-call probability from among four output base-call probabilities as a nucleobase call for the target cluster of oligonucleotides and the target sequencing cycle.

In addition to such base-call probabilities, in some embodiments, the softmax layer 514 comprises a unit-wise softmax classification layer that outputs multiple base-call-probability sets corresponding to different oligonucleotide clusters, as described by Jaganathan. Accordingly, for a given target sequencing cycle, in some embodiments, the softmax layer 514 outputs nucleobase calls for multiple target oligonucleotide clusters.

As further suggested by FIG. 5A, the adaptive sequencing system 104 can iterate through a forward path for the base-calling-neural network 500 to determine nucleobase calls for some (or all) oligonucleotide clusters captured by images for all sequencing cycles of a sequencing run. By iteratively inputting feature maps representing oligonucleotide-cluster images, passing the feature maps through the layers of the base-calling-neural network 500, and outputting base-call probabilities, the adaptive sequencing system 104 can determine nucleobase calls for nucleotide-fragment reads of a sequencing run.

As depicted in FIG. 5A, the architecture of the base-calling-neural network 500 is merely an example. In some cases, for instance, a base-calling-neural network may include non-linear layers. Further, the base-calling-neural network may include layers that perform both spatial and temporal convolution operations or layers that perform temporal and linear convolution operations. Likewise, the base-calling-neural network can include any layers or architecture described by Jaganathan.

After running a forward path of the base-calling-neural network 500 and determining one or more nucleobase calls, in some embodiments, the adaptive sequencing system 104 determines an error signal from the one or more nucleobase calls and a gradient for the error signal, consistent with the disclosure above. Based on the gradient, the adaptive sequencing system 104 modifies certain network parameters of a subset of layers within the base-calling-neural network 500 in a limited or truncated backward path. In accordance with one or more embodiments, FIG. 5B depicts the adaptive sequencing system 104 modifying certain network parameters for a subset of layers within the base-calling-neural network 500 as part of a backward path.

As shown in FIG. 5B, the adaptive sequencing system 104 freezes or maintains network parameters for a subset of spatial layers 518 within the base-calling-neural network 500. For instance, while back propagating through other layers of the base-calling-neural network 500, the adaptive sequencing system 104 maintains fixed weights for the subset of spatial layers 518, where the weights were initially learned offline using a different computing device (e.g., remote computing device in a data center). Because spatial convolutions from the set of spatial layers 508 consume considerable processing power, as explained further below, the adaptive sequencing system 104 saves or avoids consuming such processing power when modifying network parameters of a subset of layers during or after a sequencing run on a sequencing device.

By contrast, as further shown in FIG. 5B, the adaptive sequencing system 104 modifies weights (or other network parameters) of a subset of spatial layers 520 and the set of temporal layers 510 as part of a backward path. By selectively modifying weights or other parameters of the subset of spatial layers 520 and the set of temporal layers 510 as part of a backward path, in some embodiments, the adaptive sequencing system 104 selectively trains network parameters for a subset of layers of the base-calling-neural network 500. Indeed, as the adaptive sequencing system 104 modifies network parameters during or after different sequencing cycles, the adaptive sequencing system 104 can likewise track and store changes to weights or other network parameters. In some embodiments, the adaptive sequencing system 104 selectively modifies network parameter during or after intervals of sequencing cycles, such as by selectively modifying network parameters at every 10, 20, 50, or 100 sequencing cycles. Consistent with the disclosure above, in some embodiments, the adaptive sequencing system 104 selectively modifies a subset(s) of weights or a subset(s) of scaling values within the subset of spatial layers 520 and/or within the set of temporal layers 510. Such subset(s) or weights or scaling values may be assigned to respective subregions within oligonucleotide-cluster images.

As suggested above, the adaptive sequencing system 104 can extemporaneously modify such a subset of network parameters during or after a sequencing run on a sequencing device. Accordingly, after a sequencing device uses the base-calling-neural network 500 to determine one or more nucleobase calls in a forward path for a target sequencing cycle, the adaptive sequencing system 104 can modify certain network parameters based on a gradient as part of a backward path-thereby improving an accuracy of oligonucleotide-cluster-image analysis and nucleobase calls during the sequencing run. Unlike conventional training methods, the adaptive sequencing system 104 does not need or perform batch normalization for fine tuning of selected network parameters because, in some cases, batch normalized weights are folded into linear layers, such as the set of spatial layers 508 and/or the set of temporal layers 510, during back propagation. By configuring a configurable processor to implement the base-calling-neural network 500 and adjusting a subset of network parameters for a subset of layers during a sequencing run, the adaptive sequencing system 104 avoids the computational costs of adjusting a full set of parameters in each iteration and thereby saves time and processing for continuing a sequencing run.

While FIG. 5B depicts the adaptive sequencing system 104 modifying only certain network parameters, in some embodiments, the adaptive sequencing system 104 modifies network parameters for both the set of spatial layers 508 and the set of temporal layers 510. For instance, after completing a sequencing run, in certain implementations, the adaptive sequencing system 104 determines an error signal and gradient based on the nucleobase calls of the sequencing run and subsequently modifies a full set of weights within both the set of spatial layers 508 and the set of temporal layers 510 based on the gradient.

As suggested above, in some embodiments, the adaptive sequencing system 104 can determine (and store) intermediate values output by layers of a neural network as part of a forward path for determining a predicted class for a target time period. For subsequent or otherwise different time periods, the adaptive sequencing system 104 can subsequently reuse the intermediate values to determine predicted classes. In accordance with one or more embodiments, FIG. 5C illustrates the adaptive sequencing system 104 (i) determining nucleobase calls for oligonucleotide-cluster images of a target sequencing cycle based on intermediate values for an additional oligonucleotide-cluster image corresponding to an additional sequencing cycle and (ii) generating additional nucleobase calls for the additional oligonucleotide-cluster image and the additional sequencing cycle based on the intermediate values. While the following description of FIG. 5C focuses on a base-calling-neural network, a sequencing device, and nucleobase calls, the adaptive sequencing system 104 can perform the same type of actions described below with respect to another neural network, computing devices, and predicted class(es).

As shown in FIG. 5C, among various input oligonucleotide-cluster images, the adaptive sequencing system 104 inputs an image 524 of oligonucleotide clusters for an additional sequencing cycle into the base-calling-neural network 500 to determine one or more nucleobase calls 528 for a target sequencing cycle. As indicated above, in some embodiments, the image 524 comprises (i) a prior-cycle image of oligonucleotide clusters for a prior sequencing cycle before a target sequencing cycle or (ii) a subsequent-cycle image of oligonucleotide clusters for a subsequent cycle after the target sequencing cycle. Consistent with the disclosure above, the adaptive sequencing system 104 passes the image 524 of oligonucleotide clusters through a spatial-layer path 508a to determine intermediate values 522 (e.g., as part of a feature map output by the spatial-layer path 508a). In some embodiments, the spatial-layer path 508a is one path among the set of spatial layers 508 comprising the same weights as other parallel spatial-layer paths.

After determining the intermediate values 522 for the image 524 as a basis for nucleobase calls for a target sequencing cycle, the adaptive sequencing system 104 stores the intermediate values 522 for later use in determining nucleobase calls for a subsequent sequencing cycle. For instance, in some embodiments, the image 524 for oligonucleotide clusters corresponds to a fifth sequencing cycle. Consistent with the disclosure above and FIG. 5A, the adaptive sequencing system 104 inputs the image 524 for oligonucleotide clusters corresponding to the fifth sequencing cycle into a spatial-layer path and other oligonucleotide-cluster images corresponding to different sequencing cycles into different spatial-layer paths. Based on the input oligonucleotide-cluster images, the spatial-layer paths of by the set of spatial layers 508 output intermediate values for oligonucleotide-cluster images corresponding to both prior and subsequent sequencing cycles relevant to a third sequencing cycle (as the target sequencing cycle). Based on the intermediate values for such different oligonucleotide-cluster images, the adaptive sequencing system 104 further uses the set of temporal layers 510 to determine feature maps and base-call probabilities for a target oligonucleotide cluster and the third sequencing cycle. When the adaptive sequencing system 104 determines base-call probabilities for the target oligonucleotide cluster at a fourth sequencing cycle-rather than repass the image 524 for oligonucleotide clusters corresponding to the fifth sequencing cycle into a spatial-layer path to again redetermine the intermediate values 522—the adaptive sequencing system 104 stores and reuses the intermediate values 522. For instance, the adaptive sequencing system 104 stores the intermediate values 522 in a solid-state drive (SSD) or other suitable memory on a sequencing device.

In addition to storing the intermediate values 522, as further depicted in FIG. 5C, the adaptive sequencing system 104 compresses channels from the spatial-layer path 508a into compressed channels 521. For instance, in some embodiments, the adaptive sequencing system 104 compresses fourteen channels within the spatial-layer path 508a into two compressed channels. The intermediate values 522 from two compressed channels approximately represent the light intensities captured by the image 524 of oligonucleotide clusters. By compressing the channels for a spatial-layer path, the adaptive sequencing system 104 avoids repassing or reprocessing an oligonucleotide-cluster image through multiple layers within the set of spatial layers 508 for each sequencing cycle. Indeed, a compression from fourteen channels to two channels for a spatial-layer path reduces spatial convolutional calculations by approximately 80%.

As indicated by FIG. 5C, for instance, the adaptive sequencing system 104 uses the spatial-layer path 508a to determine the intermediate values 522 for the image 524 of oligonucleotide clusters corresponding to a sequencing cycle 526c as a basis for determining the one or more nucleobase calls 528 for a sequencing cycle 526a and further compresses channels from the spatial-layer path 508a into the compressed channels 521. After a delay of one sequencing cycle for four consecutive sequencing cycles, the adaptive sequencing system 104 reuses the intermediate values 522 and the compressed channels 521 as a basis for determining nucleobase calls for sequencing cycles 526b, 526c, 526d, and 526e. In each of the sequencing cycles 526a-526e, therefore, the base-calling-neural network 500 reuses the intermediate values 522 as one of several inputs for the set of spatial layers 508 to determine base-call probabilities.

To further increase the computing efficiency of a forward path through the base-calling-neural network 500, in some embodiments, the adaptive sequencing system 104 quantizes weights for the set of spatial layers 508 and/or the set of temporal layers 510 from higher resolution to lower resolution for the forward path. Accordingly, the adaptive sequencing system 104 can determine and modify high-resolution weights for the set of spatial layers 508 and/or the set of temporal layers 510 for back propagation. But during a forward path to determine nucleobase calls, the adaptive sequencing system 104 determines and uses a lower resolution version of the same weights through the set of spatial layers 508 and/or the set of temporal layers 510. Indeed, the lower resolution version of the weights can be applied or stored as a few bits (e.g., 3-5 bits).

As the description above indicates, the adaptive sequencing system 104 can (i) determine nucleobase calls for oligonucleotide-cluster images of a target sequencing cycle based on intermediate values for prior or subsequent oligonucleotide-cluster images corresponding to a prior or subsequent sequencing cycle and (ii) generate additional nucleobase calls for prior or subsequent oligonucleotide-cluster images corresponding to the prior or subsequent sequencing cycle based on the intermediate values.

In part because the weights of parallel spatial-layer paths in the base-calling-neural network 500 are the same, the adaptive sequencing system 104 can modify a subset of network parameters for the set of spatial layers 508 without disrupting a sequencing run. When the base-calling-neural network 500 uses the spatial-layer path 508a to initially determine the intermediate values 522, the adaptive sequencing system 104 can modify or incorporate adjusted weights into the spatial-layer path 508a. Because the same weights of the spatial-layer path 508a would be reused to redetermine the intermediate values 508a—if the adaptive sequencing system 104 were to repass the image 524 for oligonucleotide clusters through the spatial-layer path 508a to again redetermine the intermediate values 522—the adaptive sequencing system 104 can modify weights for a subset of layers from the set of spatial layers 508 without disrupting the forward path of the base-calling-neural network 500. To account for the iterative or repeat spatial-layer paths of the base-calling-neural network 500, in some embodiments, the adaptive sequencing system 104 modifies network parameters for the subset of spatial layers 520 in between sequencing runs.

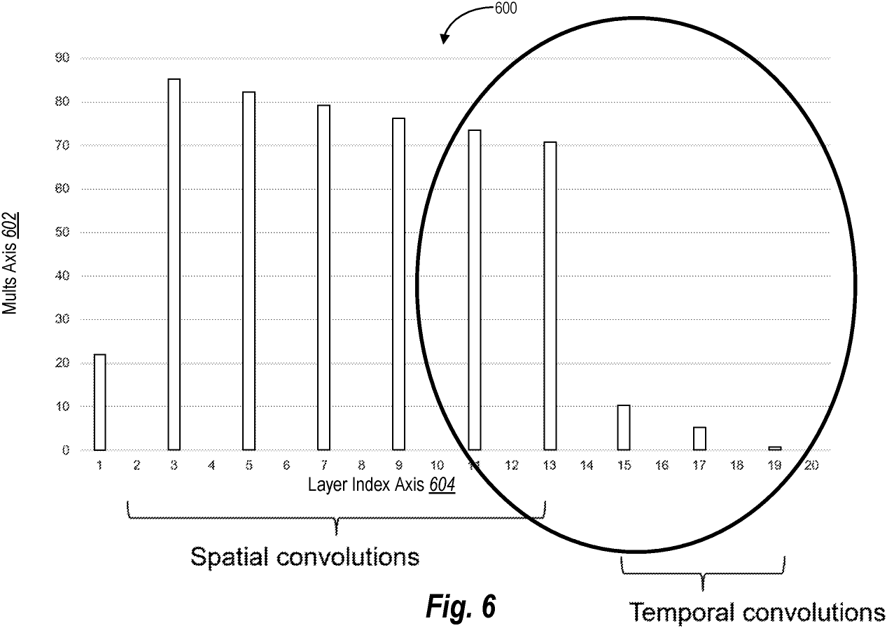

As indicated above, in some embodiments, the adaptive sequencing system 104 reduces the computer processing required for training a neural network over conventional sequencing systems and thereby increases computational efficiency. In accordance with one or more embodiments, FIG. 6 illustrates a graph 600 depicting multiplication operations (or “mults”) of a processor per image-patch operation for convolutional operations corresponding to different layers of a base-calling-neural network. As indicated by the graph 600, the multiplication operations for processing an image patch through a set of spatial layers significantly exceeds the multiplication operations for processing the image patch through a set of temporal layers within the disclosed base-calling-neural network—for both a forward path of implementing the base-calling-neural network and a backward path for adjusting network parameters of the base-calling-neural network.

As shown in the graph 600, a mults axis 602 depicts a number of million multiplication operations by a computer processor per image-patch operation for a 100×100 patch of pixels for each layer within an embodiment of the base-calling-neural network. By contrast, a layer index axis 604 depicts an individual number for each of 20 layers within the embodiment of the base-calling-neural network. As indicated by the layer index axis 604, network layers 2-13 comprise a set of spatial layers, and network layers 15-19 comprise a set of spatial layers. As further indicated by the graph 600, multiplication operations required by spatial convolutions of the set of spatial layers exceed multiplication operations required by temporal convolutions of the set of temporal layers by tens of millions. Indeed, the multiplication operations required by spatial convolutions of the set of spatial layers consume approximately 97% of total computer processing for the embodiment of the base-calling-neural network represented by FIG. 6.

Accordingly, the graph 600 demonstrates that, in some cases, the adaptive sequencing system 104 uses less computer processing for both forward and backward paths of a base-calling-neural network in comparison to conventional neural networks for base calls. By compressing channels of spatial-layer paths from 14 channels to 2 compress channels, for instance, the adaptive sequencing system 104 considerably reduces the millions of multiplication operations required to process an image patch of oligonucleotide clusters through a set of spatial layers. By back propagating a gradient to adjust a subset of network parameters for the set of temporal layers and a subset of spatial layers, as a further example, the adaptive sequencing system 104 considerably reduces the millions of multiplication operations required to train or back propagate parameters in comparison to a conventional neural network that modifies a full set of parameters in a full set of layers.

As further indicated above, in some embodiments, the adaptive sequencing system 104 performs a selective modification of network parameters for a base-calling-neural network that improves an accuracy of oligonucleotide-cluster-image analysis and base calling relative to existing neural networks for base calling. In accordance with one or more embodiments, FIG. 7 illustrates a graph 702 depicting base-call error rates for a sequencing device running a first version of a base-calling-neural network with fixed network parameters and a sequencing device running a second version of a base-calling-neural network with network parameters adjusted by the adaptive sequencing system 104 during a sequencing run.

To test the accuracy of different versions of a base-calling-neural network, researchers used a NextSeq® sequencing device implementing a first version of a base-calling-neural network with fixed network parameters and the NextSeq®™ sequencing device running a second version of a base-calling-neural network with network parameters adjusted by the adaptive sequencing system 104. As indicated by a row for “Adaptive + 3×3subregions” in a chart 700, the adaptive sequencing system 104 modifies network parameters for a subset of layers of the second version of the base-calling-neural network corresponding to a subregion (e.g., subtile) of a nucleotide-sample slide represented by an image patch divided into 3 × 3 subregions of a tile on the nucleotide-sample slide. As indicated by a row for “Baseline” in the chart 700, the adaptive sequencing system 104 does not modify the first version of the base-calling-neural network. In both cases, however, the sequencing device uses the base-calling-neural network to determine nucleobase calls for a well-known genome, PhiX.

As shown in the graph 702, a vertical axis 704a comprises base-call error rates for both the first and second versions of the base-calling-neural network across different sequencing cycles. By contrast, the horizontal axis 704b comprises numerical values indicating a number for a particular sequencing cycle. For the test depicted in the graph 702, the adaptive sequencing system 104 modifies certain network parameters for a subset of layers in the second version of the base-calling-neural network on sequencing cycle 10 and sequencing cycle 30 of both non-index nucleotide-fragment reads. As indicated above, the adaptive sequencing system 104 can likewise select different sequencing cycles or different internals of sequencing cycles at which to modify certain network parameters of a base-calling-neural network, thereby training network parameters for a subset of layers of a base-calling-neural network at selected sequencing cycles. As indicated by the graph 702, the second version of the base-calling-neural network exhibits a lower base-call error rate across sequencing cycles than the first version of the base-calling-neural network.

Indeed, as shown by the chart 700, the second version of the base-calling-neural network exhibits an approximately 9.4% improvement in median base-call error rate (i.e., 0.201% for “Adaptive + 3×3subregions” compared to 0.222% for “Baseline”) across sequencing cycles compared to the first version of the base-calling-neural network. As further indicated by the chart 700, the second version of the base-calling-neural network also exhibits an approximately 0.60% improvement in the rate of nucleobase calls for nucleotide-fragment reads passing a quality filter (i.e., 75.75% PF for “Adaptive + 3×3subregions” compared to 75.15% PF for “Baseline”) compared to the first version of the base-calling-neural network. By increasing the percentage of PF, the second version of the base-calling-neural network increases a total number of clusters considered increases at the same time that the median base-call error rate decreased. As indicated by the chart 700, the adaptive sequencing system 104 can significantly improve base-call error rate of a base-calling-neural network by modifying weights of a selected subset of layers of the base-calling-neural network. In addition to error-rate improvements, both the first and second versions of the base-calling-neural network demonstrate a same “aligned” rate shown in the chart 700, which indicates a percentage of a genomic sample that aligned to the PhiX genome as determined for each level or nucleotide-fragment read independently.

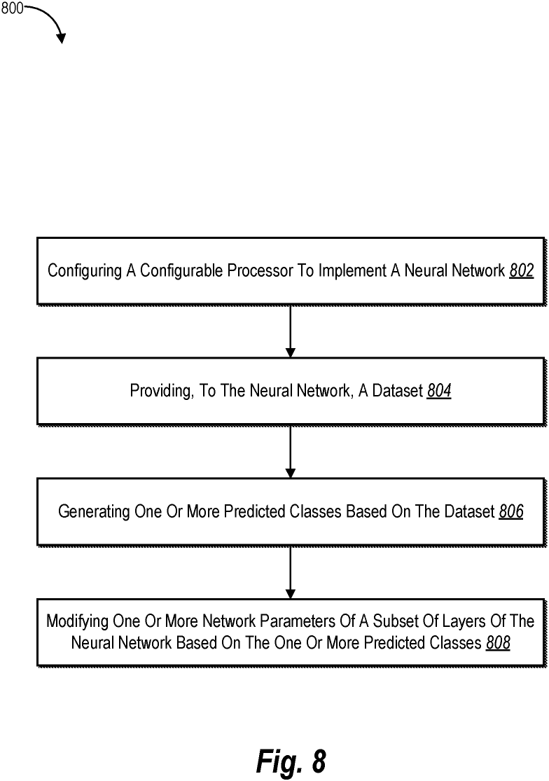

Turning now to FIG. 8, this figure illustrates a flowchart of a series of acts 800 of configuring a configurable processor to implement a neural network and train the neural network using the configurable processor by adjusting one or more network parameters of a subset of the neural network’s layers in accordance with one or more embodiments of the present disclosure. While FIG. 8 illustrates acts according to one embodiment, alternative embodiments may omit, add to, reorder, and/or modify any of the acts shown in FIG. 8. The acts of FIG. 8 can be performed as part of a method. Alternatively, a non-transitory computer readable storage medium can comprise instructions that, when executed by one or more processors, cause a computing device or a system to perform the acts depicted in FIG. 8. In still further embodiments, a system comprising at least one processor and a non-transitory computer readable medium comprising instructions that, when executed by one or more processors, cause the system to perform the acts of FIG. 8.

As shown in FIG. 8, the acts 800 include an act 802 of configuring a configurable processor to implement a neural network. In particular, in some embodiments, the act 802 includes configuring a configurable processor to implement a neural network comprising a first set of layers and a second set of layers that were initially trained using training datasets. In one or more embodiments, the configurable processor comprises an application-specific integrated circuit (ASIC), an application-specific standard product (ASSP), a coarse-grained reconfigurable array (CGRA), or a field programmable gate array (FPGA). For example, the act 802 can include configuring a field programmable gate array (FPGA) to implement a neural network comprising a first set of layers and a second set of layers that were initially trained using training datasets. In certain embodiments, the first set of layers comprise a set of bottom layers and the second set of layers comprise a set of top layers.

As suggested above, in one or more embodiments, configuring the configurable processor comprises configuring the configurable processor to implement the neural network on one or more computing devices of a system differing from one or more additional computing devices of a different system used to initially train the neural network using the training datasets.

As further shown in FIG. 8, the acts 800 include an act 804 of providing, to the neural network, a dataset. In particular, in some embodiments, the act 804 includes providing the dataset by inputting a prior-period image corresponding to a prior time period, a target-period image corresponding to a target time period, and a subsequent-period image corresponding to a subsequent time period.

As further shown in FIG. 8, the acts 800 include an act 806 of generating one or more predicted classes based on the dataset. In particular, in certain implementations, the act 806 includes generating, utilizing the neural network, one or more predicted classes based on the dataset. As suggested above, in some embodiments, generate the one or more predicted classes for the target time period based on the prior-period image, the target-period image, and the subsequent-period image.

As further shown in FIG. 8, the acts 800 include an act 808 of modifying one or more network parameters of a subset of layers of the neural network based on the one or more predicted classes. In particular, in certain implementations, the act 808 includes modifying, utilizing the configurable processor, one or more network parameters of the second set of layers based on the one or more predicted classes. As suggested above, in some embodiments, the act 808 includes modify, utilizing the FPGA, one or more network parameters of the second set of layers based on the one or more predicted classes.

Further, in some cases, modifying the one or more network parameters of the second set of layers comprises: determining a gradient with a fixed-point range based on an error signal derived from the one or more predicted classes; and adjusting one or more weights for one or more layers of the second set of layers according to the determined gradient. Relatedly, in some cases, modifying the one or more network parameters of the second set of layers comprises: identifying, from the second set of layers, subsets of weights or subsets of scaling values assigned to respective subregions within images of the dataset; and modifying the subsets of weights or the subsets of scaling values based on the one or more predicted classes.

In addition to the acts 802-808, in certain implementations, the acts 800 further include modifying the one or more network parameters of a first subset of layers from the first set of layers based on the one or more predicted classes without modifying network parameters of a second subset of layers from the first set of layers. By contrast, in certain implementations, the acts 800 further include modifying one or more network parameters of the second set of layers based on the one or more predicted classes. Additionally, or alternatively, in some embodiments, generating the one or more predicted classes for the target time period comprises determining, using the first set of layers, intermediate values for the subsequent-period image; and generating one or more additional predicted classes for the subsequent time period comprises reusing the intermediate values for the subsequent-period image.

As further suggested above, in certain cases, the acts 800 further include generating, utilizing an additional instance of the neural network implemented by an additional configurable processor, one or more additional predicted classes based on an additional dataset; and modifying, utilizing the configurable processor, a subset of network parameters of the second set of layers from the neural network based on the one or more additional predicted classes.

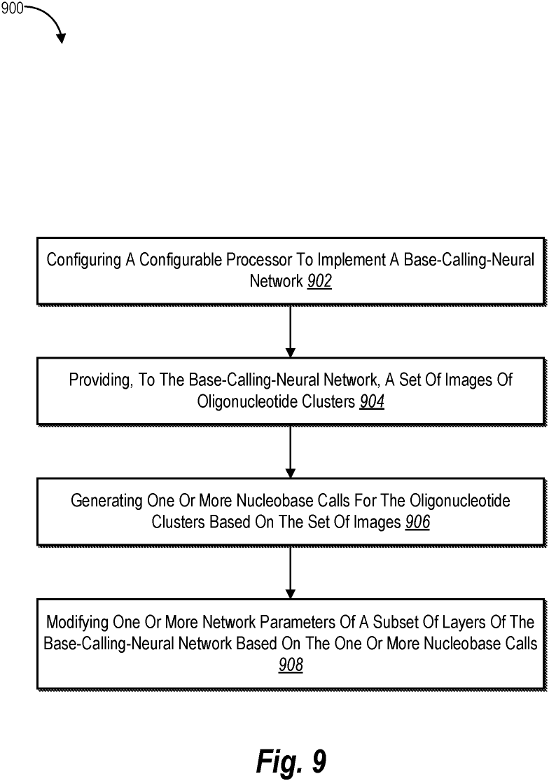

Turning now to FIG. 9, this figure illustrates a flowchart of a series of acts 900 of receiving nucleotide-sample slide comprising calibration sequences and determining one or more sequencing parameters corresponding to a sequencing device based on the calibration sequences in accordance with one or more embodiments of the present disclosure. While FIG. 9 illustrates acts according to one embodiment, alternative embodiments may omit, add to, reorder, and/or modify any of the acts shown in FIG. 9. The acts of FIG. 9 can be performed as part of a method. Alternatively, a non-transitory computer readable storage medium can comprise instructions that, when executed by one or more processors, cause a computing device or a system to perform the acts depicted in FIG. 9. In still further embodiments, a system comprising at least one processor and a non-transitory computer readable medium comprising instructions that, when executed by one or more processors, cause the system to perform the acts of FIG. 9.

As shown in FIG. 9, the acts 900 include an act 902 of configuring a configurable processor to implement a base-calling-neural network. In particular, in some embodiments, the act 902 includes configuring a configurable processor to implement a base-calling-neural network comprising a set of bottom layers and a set of top layers that were initially trained using training images of oligonucleotide clusters. In one or more embodiments, the configurable processor comprises an application-specific integrated circuit (ASIC), an application-specific standard product (ASSP), a coarse-grained reconfigurable array (CGRA), or a field programmable gate array (FPGA). For example, the act 902 can include configuring a field programmable gate array (FPGA) to implement a base-calling-neural network comprising a set of bottom layers and a set of top layers that were initially trained using training images of oligonucleotide clusters. Relatedly, in some cases, the set of bottom layers comprises a set of spatial layers and the set of top layers comprises a set of temporal layers.

As suggested above, in one or more embodiments, configuring the configurable processor comprises configuring the configurable processor to implement the base-calling-neural network on one or more computing devices of the system differing from one or more additional computing devices of a different system used to initially train the base-calling-neural network using the training images.

As further shown in FIG. 9, the acts 900 include an act 904 of providing, to the base-calling-neural network, a set of images of oligonucleotide clusters. In particular, in some embodiments, the act 904 includes providing, to the base-calling-neural network, a set of images of oligonucleotide clusters associated with a target sequencing cycle.

As further shown in FIG. 9, the acts 900 include an act 906 of generating one or more nucleobase calls for the oligonucleotide clusters based on the set of images. In particular, in certain implementations, the act 906 includes generating, utilizing the base-calling-neural network, one or more nucleobase calls for the oligonucleotide clusters and the target sequencing cycle based on the set of images.

Further, in some cases, generating the one or more nucleobase calls comprises: determining, utilizing the set of top layers, a set of output values corresponding to the set of images; and determining, utilizing a softmax layer, base-call probabilities for different nucleobase classes based on the set of output values.

As further shown in FIG. 9, the acts 900 include an act 908 of modifying one or more network parameters of a subset of layers of the base-calling-neural network based on the one or more nucleobase calls. In particular, in certain implementations, the act 908 includes modifying, utilizing the configurable processor, one or more network parameters of the set of top layers based on the one or more nucleobase calls. As suggested above, in some embodiments, the act 908 includes modifying, utilizing the FPGA, one or more network parameters of the set of top layers based on the one or more nucleobase calls.

As suggested above, in some embodiments, modifying the one or more network parameters of the set of top layers comprises: determining a gradient with a fixed-point range based on an error signal derived from the one or more nucleobase calls; and adjusting one or more weights for one or more top layers of the set of top layers according to the determined gradient. Relatedly, in some cases, modifying the one or more network parameters of the set of top layers comprises: identifying, from the set of top layers, subsets of weights or subsets of scaling values assigned to respective subregions within images of the set of images; and modifying the subsets of weights or the subsets of scaling values based on the one or more nucleobase calls. As suggested above, in some cases, the respective subregions within images represent subtiles of a tile from a nucleotide-sample slide.

Further, in some embodiments, the acts 900 include providing the set of images of oligonucleotide clusters associated with the target sequencing cycle by inputting a prior-cycle image of the oligonucleotide clusters for a prior sequencing cycle before the target sequencing cycle, a target-cycle image of the oligonucleotide clusters for the target sequencing cycle, and a subsequent-cycle image of the oligonucleotide clusters for a subsequent sequencing cycle after the target sequencing cycle; and generating the one or more nucleobase calls for the target sequencing cycle based on the prior-cycle image, the target-cycle image, and the subsequent-cycle image.

As further suggested above, in one or more implementations, the acts 900 include generating the one or more nucleobase calls for the oligonucleotide clusters and the target sequencing cycle in part by determining, using the set of bottom layers, intermediate values for the subsequent-cycle image; and generating one or more additional nucleobase call for the oligonucleotide clusters and the subsequent sequencing cycle in part by reusing the intermediate values for the subsequent-cycle image.

Further, in some embodiments, the acts 900 include generating, utilizing an additional instance of the base-calling-neural network implemented by an additional configurable processor, one or more additional nucleobase calls for additional oligonucleotide clusters and a sequencing cycle based on an additional set of images; and modifying, utilizing the configurable processor, a subset of network parameters of the set of top layers from the base-calling-neural network based on the one or more additional nucleobase calls.

In addition to the acts 902-908, in certain implementations, the acts 900 further include modifying one or more network parameters of a first subset of bottom layers from the set of bottom layers based on the one or more nucleobase calls without modifying network parameters of a second subset of bottom layers from the set of bottom layers. By contrast, in some cases, the acts 900 further include modifying one or more network parameters of the set of bottom layers based on the one or more nucleobase calls.

As further suggested above, in some embodiments, the acts 900 further include configuring the configurable processor on a sequencing device to implement the base-calling-neural network; and modifying, utilizing the configurable processor on the sequencing device, the one or more network parameters of the set of top layers based on the one or more nucleobase calls.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568 and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. No. 7,427,673, and U.S. Pat. No. 7,057,026, the disclosures of which are incorporated herein by reference in their entireties.

Additional exemplary SBS systems and methods which can be utilized with the methods and systems described herein are described in U.S. Pat. Application Publication No. 2007/0166705, U.S. Pat. Application Publication No. 2006/0188901, U.S. Pat. No. 7,057,026, U.S. Pat. Application Publication No. 2006/0240439, U.S. Pat. Application Publication No. 2006/0281109, PCT Publication No. WO 05/065814, U.S. Pat. Application Publication No. 2005/0100900, PCT Publication No. WO 06/064199, PCT Publication No. WO 07/010,251, U.S. Pat. Application Publication No. 2012/0270305 and U.S. Pat. Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Pat. Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pat. Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. No. 6,969,488, U.S. Pat. No. 6,172,218, and U.S. Pat. No. 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. No. 7,329,492 and U.S. Pat. No. 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Pat. Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeqTM platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture microdissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the adaptive sequencing system 104 can include software, hardware, or both. For example, the components of the adaptive sequencing system 104 can include one or more instructions stored on a computer-readable storage medium and executable by processors of one or more computing devices (e.g., the client device 114). When executed by the one or more processors, the computer-executable instructions of the adaptive sequencing system 104 can cause the computing devices to perform the bubble detection methods described herein. Alternatively, the components of the adaptive sequencing system 104 can comprise hardware, such as special purpose processing devices to perform a certain function or group of functions. Additionally, or alternatively, the components of the adaptive sequencing system 104 can include a combination of computer-executable instructions and hardware.

Furthermore, the components of the adaptive sequencing system 104 performing the functions described herein with respect to the adaptive sequencing system 104 may, for example, be implemented as part of a stand-alone application, as a module of an application, as a plug-in for applications, as a library function or functions that may be called by other applications, and/or as a cloud-computing model. Thus, components of the adaptive sequencing system 104 may be implemented as part of a stand-alone application on a personal computing device or a mobile device. Additionally, or alternatively, the components of the adaptive sequencing system 104 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of a computing device 1000 that may be configured to perform one or more of the processes described above. One will appreciate that one or more computing devices such as the computing device 1000 may implement the adaptive sequencing system 104 and the sequencing system 112. As shown by FIG. 10, the computing device 1000 can comprise a processor 1002, a memory 1004, a storage device 1006, an I/O interface 1008, and a communication interface 1010, which may be communicatively coupled by way of a communication infrastructure 1012. In certain embodiments, the computing device 1000 can include fewer or more components than those shown in FIG. 10. The following paragraphs describe components of the computing device 1000 shown in FIG. 10 in additional detail.

In one or more embodiments, the processor 1002 includes hardware for executing instructions, such as those making up a computer program. As an example, and not by way of limitation, to execute instructions for dynamically modifying workflows, the processor 1002 may retrieve (or fetch) the instructions from an internal register, an internal cache, the memory 1004, or the storage device 1006 and decode and execute them. The memory 1004 may be a volatile or non-volatile memory used for storing data, metadata, and programs for execution by the processor(s). The storage device 1006 includes storage, such as a hard disk, flash disk drive, or other digital storage device, for storing data or instructions for performing the methods described herein.

The I/O interface 1008 allows a user to provide input to, receive output from, and otherwise transfer data to and receive data from computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1008 is configured to provide graphical data to a display for presentation to a user. The graphical data may be representative of one or more graphical user interfaces and/or any other graphical content as may serve a particular implementation.

The communication interface 1010 can include hardware, software, or both. In any event, the communication interface 1010 can provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium comprising instructions that, when executed by the at least one processor, cause the system to:
configure a field programmable gate array (FPGA) to implement a base-calling-neural network comprising a set of bottom layers and a set of top layers that were initially trained using training images of oligonucleotide clusters;
provide, to the base-calling-neural network, a set of images of oligonucleotide clusters associated with a target sequencing cycle;
generate, utilizing the base-calling-neural network, one or more nucleobase calls for the oligonucleotide clusters and the target sequencing cycle based on the set of images; and
modify, utilizing the FPGA, one or more network parameters of the set of top layers based on the one or more nucleobase calls.

2. The system of claim 1, wherein the set of bottom layers comprises a set of spatial layers and the set of top layers comprises a set of temporal layers.

3. The system of claim 1, further comprising instructions that, when executed by the at least one processor, cause the system to modify the one or more network parameters of the set of top layers by:
determining a gradient with a fixed-point range based on an error signal derived from the one or more nucleobase calls; and
adjusting one or more weights for one or more top layers of the set of top layers according to the determined gradient.

4. The system of claim 1, further comprising instructions that, when executed by the at least one processor, cause the system to modify one or more network parameters of a first subset of bottom layers from the set of bottom layers based on the one or more nucleobase calls without modifying network parameters of a second subset of bottom layers from the set of bottom layers.

5. The system of claim 1, further comprising instructions that, when executed by the at least one processor, cause the system to configure the FPGA to implement the base-calling-neural network on one or more computing devices of the system differing from one or more additional computing devices of a different system used to initially train the base-calling-neural network using the training images.

6. The system of claim 1, further comprising instructions that, when executed by the at least one processor, cause the system to modify the one or more network parameters of the set of top layers by:
identifying, from the set of top layers, subsets of weights or subsets of scaling values assigned to respective subregions within images of the set of images; and
modifying the subsets of weights or the subsets of scaling values based on the one or more nucleobase calls.

7. The system of claim 1, further comprising instructions that, when executed by the at least one processor, cause the system to:
provide the set of images of oligonucleotide clusters associated with the target sequencing cycle by inputting a prior-cycle image of the oligonucleotide clusters for a prior sequencing cycle before the target sequencing cycle, a target-cycle image of the oligonucleotide clusters for the target sequencing cycle, and a subsequent-cycle image of the oligonucleotide clusters for a subsequent sequencing cycle after the target sequencing cycle; and
generate the one or more nucleobase calls for the target sequencing cycle based on the prior-cycle image, the target-cycle image, and the subsequent-cycle image.

8. The system of claim 7, further comprising instructions that, when executed by the at least one processor, cause the system to:
generate the one or more nucleobase calls for the oligonucleotide clusters and the target sequencing cycle in part by determining, using the set of bottom layers, intermediate values for the subsequent-cycle image; and
generate one or more additional nucleobase call for the oligonucleotide clusters and the subsequent sequencing cycle in part by reusing the intermediate values for the subsequent-cycle image.

9. The system of claim 1, further comprising instructions that, when executed by the at least one processor, cause the system to generate the one or more nucleobase calls by:
determining, utilizing the set of top layers, a set of output values corresponding to the set of images; and
determining, utilizing a softmax layer, base-call probabilities for different nucleobase classes based on the set of output values.

10. A non-transitory computer readable medium comprising instructions that, when executed by at least one processor, cause a system to:
configure a configurable processor to implement a base-calling-neural network comprising a set of bottom layers and a set of top layers that were initially trained using training images of oligonucleotide clusters;
provide, to the base-calling-neural network, a set of images of oligonucleotide clusters associated with a target sequencing cycle;
generate, utilizing the base-calling-neural network, one or more nucleobase calls for the oligonucleotide clusters and the target sequencing cycle based on the set of images; and
modify, utilizing the configurable processor, one or more network parameters of the set of top layers based on the one or more nucleobase calls.

11. The non-transitory computer readable medium of claim 10, wherein the configurable processor comprises an application-specific integrated circuit (ASIC), an application-specific standard product (ASSP), a coarse-grained reconfigurable array (CGRA), or a field programmable gate array (FPGA).

12. The non-transitory computer readable medium of claim 10, wherein the set of bottom layers comprises a set of spatial layers and the set of top layers comprises a set of temporal layers.

13. The non-transitory computer readable medium of claim 10, further comprising instructions that, when executed by the at least one processor, cause the system to modify the one or more network parameters of the set of top layers by:
determining a gradient with a fixed-point range based on an error signal derived from the one or more nucleobase calls; and
adjusting one or more weights for one or more top layers of the set of top layers according to the determined gradient.

14. The non-transitory computer readable medium of claim 10, further comprising instructions that, when executed by the at least one processor, cause the system to modify the one or more network parameters of the set of top layers by:
identifying, from the set of top layers, subsets of weights or subsets of scaling values assigned to respective subregions within images of the set of images; and
modifying the subsets of weights or the subsets of scaling values based on the one or more nucleobase calls.

15. The non-transitory computer readable medium of claim 10, further comprising instructions that, when executed by the at least one processor, cause the system to:
provide the set of images of oligonucleotide clusters associated with the target sequencing cycle by inputting a prior-cycle image of the oligonucleotide clusters for a prior sequencing cycle before the target sequencing cycle, a target-cycle image of the oligonucleotide clusters for the target sequencing cycle, and a subsequent-cycle image of the oligonucleotide clusters for a subsequent sequencing cycle after the target sequencing cycle; and
generate the one or more nucleobase calls for the target sequencing cycle based on the prior-cycle image, the target-cycle image, and the subsequent-cycle image.

16. The non-transitory computer readable medium of claim 15, further comprising instructions that, when executed by the at least one processor, cause the system to:
generate the one or more nucleobase calls for the oligonucleotide clusters and the target sequencing cycle in part by determining, using the set of bottom layers, intermediate values for the subsequent-cycle image; and
generate one or more additional nucleobase call for the oligonucleotide clusters and the subsequent sequencing cycle in part by reusing the intermediate values for the subsequent-cycle image.

17. The non-transitory computer readable medium of claim 10, further comprising instructions that, when executed by the at least one processor, cause the system to configure the configurable processor to implement the base-calling-neural network on one or more computing devices of the system differing from one or more additional computing devices of a different system used to initially train the base-calling-neural network using the training images.

18. A computer-implemented method comprising:
configuring a configurable processor to implement a base-calling-neural network comprising a set of bottom layers and a set of top layers that were initially trained using training images of oligonucleotide clusters;
providing, to the base-calling-neural network, a set of images of oligonucleotide clusters associated with a target sequencing cycle;
generating, utilizing the base-calling-neural network, one or more nucleobase calls for the oligonucleotide clusters and the target sequencing cycle based on the set of images; and
modifying, utilizing the configurable processor, one or more network parameters of the set of top layers based on the one or more nucleobase calls.

19. The computer-implemented method of claim 18, further comprising:
generating, utilizing an additional instance of the base-calling-neural network implemented by an additional configurable processor, one or more additional nucleobase calls for additional oligonucleotide clusters and a sequencing cycle based on an additional set of images; and
modifying, utilizing the configurable processor, a subset of network parameters of the set of top layers from the base-calling-neural network based on the one or more additional nucleobase calls.

20. The computer-implemented method of claim 18, wherein modifying the one or more network parameters comprises modifying one or more network parameters of a first subset of bottom layers from the set of bottom layers based on the one or more nucleobase calls without modifying network parameters of a second subset of bottom layers from the set of bottom layers.
